(19) United States
(12) Patent Application Publication
(10) Pub. No.: US 2026/0355404 A1
(43) Pub. Date: Nov. 12, 2026

(54) Manifolds, Systems and Methods For Filtering Surgical Smoke

(71) Applicant: Stryker Corporation, Portage, MI (US)

(72) Inventors: Jacob C. Foor, Kalamazoo, MI (US); Brandon Lee Laure, Satellite Beach, FL (US); Brian MacLachlan, Norton Shores, MI (US); Noah Needler, Mattawan, MI (US); Akit Panchal, Mandoli (IN); Dinesh Sharma, Faridabad (IN); Gaurav Retwal, Faridabad (IN); Michael Zollinger, Chelsea, MI (US)

(73) Assignee: Stryker Corporation, Portage, MI (US)

(21) Appl. No.: 19/491,228

(22) PCT Filed: Dec. 6, 2024
(86) PCT No.: PCT/US2024/058892

§ 371 Date: Dec. 8, 2025

Related U.S. Application Data
(60) Provisional application No. 63/606,737, filed on Dec. 6, 2023.
(60) Provisional application No. 63/708,511, filed on Oct. 17, 2024.

(51) Int. Cl. A61M 1/00; A61B 17/00; A61B 18/00

(52) U.S. Cl. CPC A61M 1/79; A61B 18/00; A61M 1/87; A61B 2017/00738; A61B 2218/008

(57) ABSTRACT

A manifold for filter surgical smoke may include a head portion or a trunk within which a filter assembly is disposed. The head portion may include an inlet fitting and define a fluid sump. A port of a filter casing may be staggered from the inlet fitting. The filter assembly includes at least one particulate filter and/or at least one adsorber. A bypass fitting for another suction tube may be provided such that suctioned liquid medical waste bypasses the filter assembly. Systems and methods for filtering the surgical smoke may include providing an alert or preventing operation of the vacuum source if combined filtration performance between the manifold and a system filter does not meet a predefined minimum filtration performance. The combined filtration performance may be based on filter types or determined filtration ratings of the manifold and/or the system filter.

### PRIORITY CLAIM

This application claims priority to and all the benefits of U.S. Provisional Patent Application No. 63/708,511, filed Oct. 17, 2024, and U.S. Provisional Patent Application No. 63/606,737, filed Dec. 6, 2023, the entire contents of each being hereby incorporated by reference.

### BACKGROUND

Surgical smoke includes aerosolized combustion byproducts produced by heat-generating surgical instruments, such as lasers, electrosurgery, ultrasonic devices, drills, and saws. The vapors of aerosolized chemicals and volatile organic compounds (VOCs) are unpleasant and/or hazardous to health, and may include carcinogenic matter, blood and tissue particles, bacteria, viruses, odors, ash, or the like. Literature suggests that attending personnel exposed to surgical smoke report twice as many respiratory health issues as the general public. In certain instances, the smoke can be thick enough to obscure vision, especially during longer operations where cauterizing tools are heavily used. It is known to remove the surgical smoke with a smoke evacuation unit including a vacuum source, and a replaceable filter assembly. The smoke evacuation unit often is implemented as a console situated on a tabletop within the operating room.

Another byproduct of many surgical procedures is liquid medical waste, for examples, bodily fluids and irrigating solutions. It is known to remove the liquid medical waste from the surgical site under the influence of suction. One exemplary medical waste collection system for doing so is sold under the tradename Neptune by Stryker Corporation (Portage, Mich.) with certain versions of the system being disclosed in commonly-owned International Publication No. WO 2007/070570, published Jun. 21, 2007, International Publication No. WO 2014/066337, published May 1, 2014, International Publication No. WO 2017/112684, published Jun. 29, 2017, and International Publication No. WO 2020/210763, published Oct. 15, 2020, the entire contents of each being hereby incorporated by reference. A suction tube is coupled to a manifold that is removably coupled to the waste collection system. The manifold may include a filter element for filtering solid and semisolid waste material (e.g., bits of tissue) that may be entrained in the liquid medical waste. Exemplary implementations of the manifold are disclosed in commonly-owned International Publication No. WO 2007/079319, published Jul. 12, 2007, International Publication No. 2013/090579, published Jun. 20, 2013, International Publication No. WO 2019/222655, published Nov. 21, 2019, and International Publication No. WO 2020/209898, published Oct. 15, 2020, the entire contents of each being hereby incorporated by reference.

Certain medical facilities therefore undesirably require two separate systems for removal of the respective byproducts, resulting in a cumbersome workflow as well as added expense and maintenance. Furthermore, while certain models of the Neptune system include an integrated smoke evacuation unit, others do not. Therefore, there is a need in the art for systems and methods to extend the capacity of the vacuum source provided by the waste collection system to also filter surgical smoke.

The suction system may include a system filter, typically a high efficiency particulate air (HEPA) filter, for filtering air being exhausted to the operating room. However, electrosurgical devices and lasers often create particles less than 0.3 microns in size, which is too small to be effectively captured by HEPA filters. Ultra low particulate air (ULPA) filters are effective at capturing particles at 0.1 microns; however, ULPA filters suffer from several drawbacks, all of which undesirably add complexity, expense and waste. First, less air passes through an ULPA filter relative to a similarly-sized HEPA filter, and therefore a larger ULPA filter is required for a given amount of air to be filtered. Second, ULPA filters have greater pressure drop across the filter media than HEPA filters, which otherwise requires a larger vacuum source. Third, the ULPA filters themselves are costlier, which is further compounded a reduced operational lifespan due to faster clogging. Further, not all surgical procedures utilize electrocautery, and therefore it may be undesirably expensive to use an ULPA system filter at all times. Conversely, it would also be cumbersome on medical facility staff to maintain inventory of multiple filter types, and switch between the system filters based on the needs of a particular surgical procedure. Yet, in those instances, failure to do so may otherwise result in suboptimal filtration performance, which again is deleterious to the health of the attending medical personnel. Therefore, there is a further need in the art for means by which improved filtration performance can be achieved with lessened expense to the medical facility. It would be desirable do so in a generally automatic manner that is system-determined and controlled, thereby and lessening effort by the facility staff.

Often, there is trace liquid entrained within the surgical smoke (separate from any liquid medical waste being separately suctioned). A known solution includes coupling an external fluid trap to an inlet of a filter assembly. Such a solution requires an added component and associated cost, and has not been conceived on a system that provides for separate (e.g., simultaneous) suctioning of liquid medical waste. Therefore, there is an even further need in the art for a device that provides for collecting and/or filtering liquid within the surgical smoke in an improved manner.

Further advantages of the present disclosure will be readily appreciated from the written description and claims that follow, and the accompanying drawings.

### SUMMARY

Aspects of the present disclosure are directed to manifold for filtering the surgical smoke with a suction system, also referred to herein as a waste collection system. The manifold includes a particulate filter configured to filter surgical smoke. Certain implementations of the manifold disclosed herein also permit the liquid medical waste to be collected with the waste collection system, optionally, in a simultaneous manner.

The manifold includes a housing, which may be formed from a head portion rigidly or removably coupled to a trunk. The trunk defines an outlet opening configured to be inserted into a manifold receiver of the waste collection system. The trunk includes features or geometries configured to engage complementary components within the manifold receiver. A filter basket may be disposed within a body interior of the trunk. The filter basket defines apertures for capturing solid and semisolid waste material within liquid medical waste. The filter basket is not for filtering surgical smoke. The head portion defines a head interior, and an aperture defined by the head portion or the trunk provides fluid communication between the head interior and the body interior.

A filter assembly is disposed within the head interior or the trunk. The filter assembly includes a particulate filter configured to filter the surgical smoke being drawn smoke evacuation instrument through an inlet fitting configured to be removably coupled with a smoke tube coupler of a smoke evacuation instrument. For example, the particulate filter may be an ultra-low particulate air (ULPA) filter, a high-efficiency particulate air (HEPA) filter, a combination thereof, among other filter media. For example, the particulate filter may be pleated media configured to provide for a low pressure drop thereacross. The filter assembly may optionally include a prefilter layer (e.g., a coalescing prefilter), one or more adsorbers, and/or a second filter media (e.g., supplemental filter).

The particulate filter may be disposed within a filter casing. The filter casing defines a casing inlet and a casing outlet with the particulate filter disposed between the inlet and the outlet. The filter casing may include a distal barrier, and a port extending distally from the distal barrier. A proximal end of the port may open through the distal barrier to define the inlet of the filter casing, and the port further defines a bore in fluid communication therewith. The filter casing is optional, and alternatively the particulate filter may be secured directly to inner surfaces of the head portion to provide a sealed pathway therethrough. The particulate filter may be joined to or supported by the inner surfaces with an adhesive, gasket, or other suitable sealing interface or component.

In certain implementations, the head portion includes the inlet fitting. Should it be desired for the manifold not be used for surgical smoke evacuation, a fitting cap may be secured over the inlet fitting. In other implementations, the head portion defines a bore, and the filter casing includes the inlet fitting extending through the bore. In particular, the filter casing is integrally formed with the inlet fitting to provide for direct connection between the smoke tube coupler and the filter assembly.

In certain implementations, the head portion includes a bypass fitting configured to receive the liquid tube coupler of a liquid suction instrument (i.e., distinct from the smoke evacuation instrument). The bypass fitting is in fluid communication with the body interior such that the liquid medical waste being drawn through the suction instrument and through bypass fitting bypasses the filter assembly. In instances where the manifold not being used for liquid waste evacuation, another fitting cap may be secured over the bypass fitting.

In certain implementations, the manifold includes a fluid sump and means for separating the liquid medical waste from the surgical smoke. The head portion may include a lower wall or protrusion that flares downwardly from an adjacent proximal length of the lower wall. The fluid sump may be at least partially disposed below the filter casing or the particulate filter. The fluid sump may be positioned between the distal barrier of the filter casing and a cap faceplate of the head portion. The inlet fitting and the port of the filter casing may be in a staggered arrangement. A proximal end of the inlet fitting may be positioned proximal to a distal end of the port of the filter casing. Further, the distal end of the port may be positioned above, below, or lateral to the proximal end of the inlet fitting. Based on the staggered arrangement of the inlet fitting and the port, the tortuous path prevents at least most of the liquid from being drawn through the inlet of the filter assembly. The gas flow path and the liquid flow path may be merged or joined within the trunk to be drawn through the outlet opening.

In certain implementations, the head portion includes a rim defining a cavity. The cavity may be sized to a known size and/or shape of conventional smoke tube couplers to provide further securing means and/or to avoid user confusion between the inlet fitting and the bypass fitting(s). The head portion may be translucent or transparent, and the filter casing may be formed from colored material corresponding to a color associated with the coupler of the smoke suction tube.

In certain implementations, the head portion includes a barrier defining the fluid sump. The barrier may define a window or aperture, and otherwise be impermeable to liquid and gas. The first particulate filter may be supported on or above the barrier. A second filter media may be disposed within the head portion, and more particularly disposed within the aperture. The second filter media may be formed to cause a higher pressure drop to prevent excessive amounts of gas from being drawn therethrough, yet permit the trace liquid to seep therethrough to join the liquid flow path. In one non-limiting example, the second filter media is mixed media or another porous material with different filtering characteristic than the first particulate filter. The first particulate filter may be supported on a step formed by the barrier, and the second filter media is disposed within the aperture on the distal face.

The barrier may include a ramped surface with the first particulate filter supported on an upper partition spaced above the barrier to form a liquid collection compartment within the head portion. A sorbent may be disposed within the liquid collection compartment. A baffle may extend downwardly from an upper interior surface of the head portion and positioned between the inlet fitting and the filter assembly. The baffle is configured to separate the liquid from the surgical smoke for the liquid to be collected with the fluid sump.

In certain implementations, the first particulate filter and the second filter media may be coupled to or otherwise separated by an impermeable separator. The first particulate filter may be disposed above the separator, and the second filter media may be disposed the separator. The separator may be oriented horizontally or angled. The prefilter media may extend between the separator and the upper aspect of the head portion. In other words, the prefilter media may be disposed upstream of the first particulate filter, but not upstream of the second filter media.

Another aspect of the present disclosure is directed to systems and methods for ensuring adequate filtration performance (e.g., HEPA+, near-ULPA, or ULPA, low VOC), and optionally in automatic manner that is system-determined and controlled. Any one of several manifold types may be used, wherein the manifolds have differing filtration performance, features (e.g., adsorber), or other characteristics. Likewise, any one of several system filters may be operably coupled within a filter receptacle of the system, wherein the system filters have differing filtration performance, features or characteristics. Each of the manifolds and the system filters may include means for storing identification data. The means may a radiofrequency identification (RFID) tag, an optical tag, memory, or the like. The manifold identification data may include a presence or absence of the particulate filter, a presence or absence of the adsorber, presence or absence of the bypass fitting (i.e., for use with liquid waste evacuation), or the like. The system filter identification data may include at least one of a system filter type, system filter filtration rating, presence or absence of a system filter adsorber, and the like.

The system may include data readers are configured detect the manifold and the system filter being operably coupled to a respective one of the manifold receiver(s) and the filter receptacle, and receive the identification data from a data storage means coupled thereto. The data is transmitted to a controller. Determinations therefrom may be made by the controller for controlling the vacuum source and/or a vacuum regulator, and/or providing information (e.g., an alert) on a user interface supported on the cart.

In certain implementations, the one or more processors determine a manifold type based on the manifold identification data, and a system filter type based on the system filter identification data. The one or more processors compares a combination of the manifold type and the system filter type against predefined combinations accessed from memory. The predefined combinations are configured achieve a minimum smoke filtration performance. The minimum smoke filtration performance may be one of a high efficiency particulate air (HEPA) rating and an ultra low particulate air (ULPA) rating, and/or volatile organic compounds (VOC) cmissions below a defined threshold.

In certain implementations, the user interface may receive a user input indicative of anticipated usage of the waste collection system. Additionally or alternatively, the user input may indicate a desired level of filtration performance such as a filtration rating or a level of VOC emissions. The predefined combinations against which the combination is compared may be based on the user input.

One of the predefined combinations may be the presence of the particulate filter, the absence of the manifold adsorber, the system filter filtration rating being HEPA rated, and the presence of the system filter adsorber. For another example, one of the predefined combinations is an absence of the particulate filter, the presence of the manifold adsorber, the system filter filtration rating being ULPA rated, and the presence of the system filter adsorber. Additionally or alternatively, the processor determine a combined filtration performance based on the manifold filtration rating and the system filter filtration rating. The combined smoke filtration performance may be one of a HEPA rating, an ULPA rating, and/or VOC emissions below a defined threshold.

Should the filtration performance and/or VOC emissions meet a desired criteria, the controller may permit normal operation of the system. An indication of optimal filtration performance may be provided on the user interface. Conversely, should the filtration performance and/or VOC emissions not meet the desired criteria, the controller may not permit operation of the system or an alert may be provided on the user interface so the attending medical personnel are aware of the increased risk to suboptimally filtered surgical smoke. Additionally or alternatively, the controller may prevent operation of the vacuum source if the filtration performance and/or VOC emissions do not meet the desired criteria. In such instances, the user interface may receive an override input from the user in response to the presented alert, after which operation of the vacuum source is permitted.

In certain implementations, the controller may be configured control the vacuum source (and/or a vacuum regulator) based on the identification data of the manifold. In particular, the controller may be configured to operate the vacuum source and/or the vacuum regulator to one or more suction profiles based on anticipated usage of the identified type of manifold. This may be done automatically without requiring input from the user. For example, the system may include a particulate sensor configured to detect the presence of smoke, after which vacuum level may be increased to remove as much smoke as possible while monitoring the thermal constrains on the vacuum source. The present disclosure also contemplates a computer program product comprising non-transitory computer readable medium configured to be executed by one or more processors to perform the steps of any one of the methods disclosed herein.

Therefore, according to an aspect of the present disclosure, a manifold for filtering surgical smoke is provided. A trunk defines an outlet opening configured to be operably coupled with a suction system. A head portion is coupled to the trunk and including an inlet fitting configured to be removably coupled with a smoke suction tube of a smoke evacuation instrument. A filter assembly disposed is within the head portion and includes a filter casing defining a casing inlet and a casing outlet. The filter assembly further includes a particulate filter supported within the filter casing between the casing inlet and the casing outlet.

According to another aspect of the present disclosure, a manifold for filtering surgical smoke is provided. A trunk defines an outlet opening configured to be operably coupled with a suction system. A head portion is coupled to the trunk and comprising an inlet fitting configured to be removably coupled with a smoke suction tube of a smoke evacuation instrument. A filter assembly includes a filter casing defining a casing inlet and a casing outlet, a particulate filter supported within the filter casing, a distal barrier defining the casing inlet, and a port extending distally from the distal barrier. An axis defined by the inlet fitting is positioned below an axis defined by the port.

According to another aspect of the present disclosure, a manifold for filtering surgical smoke is provided. A trunk defines an outlet opening configured to be operably coupled with a suction system. A head portion is coupled to the trunk and includes an inlet fitting configured to be removably coupled with a smoke suction tube of a smoke evacuation instrument. A filter assembly includes a filter casing, and a particulate filter supported within the filter casing. The head portion includes a downwardly extending protrusion defining a fluid sump. The downwardly extending protrusion is distal to the filter casing.

According to another aspect of the present disclosure, a manifold for filtering surgical smoke is provided. A trunk includes a first leg defining an outlet opening configured to be operably coupled with a suction system, and a second leg spaced apart from the first leg by a void. A head portion is coupled to the trunk and comprising an inlet fitting configured to be removably coupled with a smoke suction tube of a smoke evacuation instrument. A filter assembly is disposed within the trunk and includes a filter casing, and a particulate filter supported within the filter casing. The filter casing defines a casing inlet and a casing outlet positioned distal to the first and second legs of the trunk.

According to another aspect of the present disclosure, a manifold for filtering surgical smoke is provided. A trunk defines an outlet opening configured to be operably coupled with a suction system. A head portion is coupled to the trunk and defines a bore. A filter assembly is disposed within the head portion and includes a filter casing, and a particulate filter disposed within the filter casing. The filter casing includes a distal barrier, an inlet fitting extending distally from the distal barrier and through the bore of the head portion. The inlet fitting configured to be removably coupled with a coupler of a smoke suction tube.

According to another aspect of the present disclosure, a manifold for filtering surgical smoke is provided. A trunk defines an outlet opening configured to be operably coupled with a suction system. A head portion is coupled to the trunk and includes a rim defining a cavity sized to receive a coupler of a smoke suction tube. A bore is defined at a base of the cavity. A filter assembly is disposed within the head portion and includes a filter casing, and a particulate filter disposed within the filter casing. The filter casing includes a distal barrier and defines an inlet port within the distal barrier and aligned with the bore of the head portion. The inlet port is configured to be removably coupled with the coupler of the smoke suction tube with the coupler disposed within the cavity.

According to another aspect of the present disclosure, a manifold for filtering surgical smoke is provided. A trunk defines an outlet opening configured to be operably coupled with a suction system. A head portion is coupled to the trunk. An inlet fitting is configured to be removably coupled with a smoke suction tube of a smoke evacuation instrument to establish a gas flow path with the outlet opening. The head portion includes a bypass fitting configured to be removably coupled with a fluid suction tube of a suction instrument to establish a liquid flow path with the outlet opening. The head portion includes a barrier defining a fluid sump and separating the gas flow path and the liquid flow path other than through an aperture defined therethrough. A filter assembly is disposed within the head portion and includes a first particulate filter disposed within the gas flow path, and a second filter media disposed within the aperture of the barrier and configured to permit liquid entrained with the gas flow path to collect within the fluid sump and scep into the liquid flow path.

According to another aspect of the present disclosure, a manifold for filtering surgical smoke is provided. A trunk defines an outlet opening configured to be operably coupled with a suction system. A head portion is coupled to the trunk. An inlet fitting is configured to be removably coupled with a smoke suction tube of a smoke evacuation instrument to establish a gas flow path with the outlet opening. The head portion includes a bypass fitting configured to be removably coupled with a fluid suction tube of a suction instrument to establish a liquid flow path with the outlet opening. The head portion includes a barrier defining a fluid sump and separating the gas flow path and the liquid flow path. A filter assembly is disposed within the head portion and comprising a first particulate filter, a second filter media positioned below the first particulate filter, and an impermeable separator disposed between the first particulate filter and the second filter media.

According to another aspect of the present disclosure, a method of controlling a waste collection system includes detecting a manifold being removably inserted within a manifold receiver. The manifold includes means for storing manifold identification data. A manifold type is determined based on the manifold identification data. The method includes detecting a system filter removably inserted within the filter receptacle. The system filter includes means for storing system filter identification data. A system filter type is determined based on the system filter identification data. A combination of the manifold type and the system filter type is compared against predefined combinations accessed from memory. The predefined combinations are configured achieve a minimum smoke filtration performance. The method includes presenting an alert on the user interface or preventing operation of the vacuum source if the combination is not one of the predefined combinations.

According to another aspect of the present disclosure, a method of controlling a waste collection system includes detecting a manifold being removably inserted within a manifold receiver. The manifold includes means for storing manifold filtration rating. A manifold type is determined based on the manifold identification data. The method includes detecting a system filter removably inserted within the filter receptacle. The system filter includes means for storing system filter filtration rating. A combined filtration performance is determined based on the manifold filtration rating and the system filter filtration rating. The method includes presenting an alert on the user interface or preventing operation of the vacuum source if the combined filtration performance does not exceed a predefined minimum smoke filtration performance.

### BRIEF DESCRIPTION OF THE FIGURES

FIG. 1 is a front perspective view of an implementation of waste collection system. Implementations of a manifold are configured to be removably inserted into a manifold receiver, and implementation of a system filter are configured to be removably inserted into a filter receptacle.

FIG. 2 is a front perspective view of another implementation of waste collection system.

FIG. 3 is a front perspective view of a first implementation of the manifold.

FIG. 4 is a rear perspective view of the manifold of FIG. 3.

FIG. 5 is a front exploded view of the manifold of FIG. 3.

FIG. 6 is a sectional elevation view of the manifold of FIG. 3 taken along section lines 6-6. A filter assembly is disposed within a head portion of the manifold.

FIG. 7 is a rear perspective view of a second implementation of the manifold.

FIG. 8 is a rear exploded view of the manifold of FIG. 7.

FIG. 9 is a sectional elevation view of the manifold of FIG. 7 taken along section lines 9-9.

FIG. 10 is a front perspective view of a third implementation of the manifold.

FIG. 11 is a front exploded view of the manifold of FIG. 10.

FIG. 12 is a sectional elevation view of the manifold of FIG. 10 taken along section lines 12-12.

FIG. 13 is a front perspective view of a fourth implementation of the manifold.

FIG. 14 is a front exploded view of the manifold of FIG. 13.

FIG. 15 is a sectional elevation view of the manifold of FIG. 13 taken along section lines 15-15.

FIG. 16 is a front perspective view of a fifth implementation of the manifold.

FIG. 17 is a front exploded view of the manifold of FIG. 16.

FIG. 18 is a sectional elevation view of the manifold of FIG. 16 taken along section lines 18-18.

FIG. 19 is a front perspective view of a sixth implementation of the manifold.

FIG. 20 is a front exploded view of the manifold of FIG. 18.

FIGS. 21 and 22 are front perspective views of variants of the manifold in which certain aspects of the filter assembly within the head portion are shown in phantom.

FIGS. 23-26 are partial sectional elevation views of additional implementations of the manifold in which at least one particulate filter is provided.

FIGS. 27-31B are partial sectional elevation views of additional implementations of the manifold in which a valve assembly is configured to selectively prevent or permit ingress of liquid and/or surgical smoke through the manifold.

FIG. 32 is an exemplary method for filtering surgical smoke with the waste collection system.

FIGS. 33-35 are schematic representations of various arrangements of subcomponents of the waste collection system to provide liquid and gas flow paths therethrough.

### DETAILED DESCRIPTION

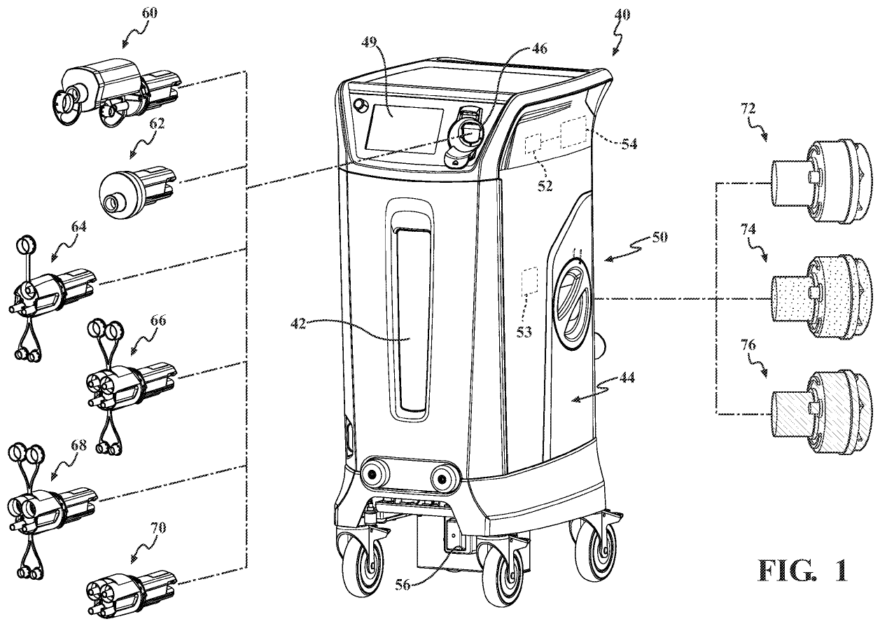

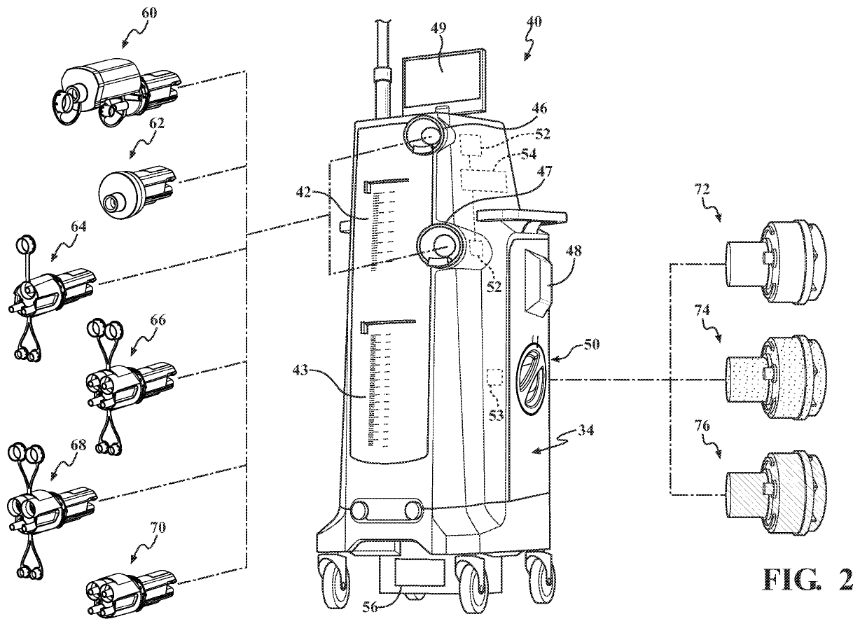

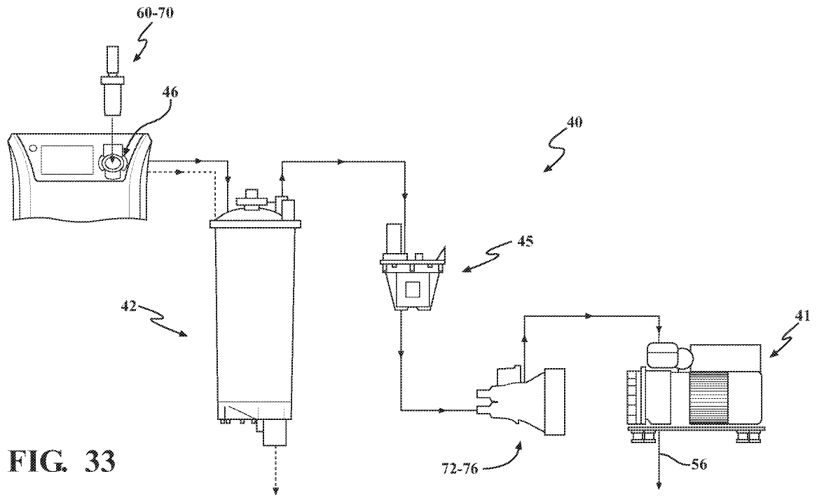

FIG. 1 shows a suction system 40 for collecting medical waste. The illustrated implementation of the suction system 40 is a waste collection system in which a waste canister 42 and a vacuum source 41 (see FIGS. 33-35) are supported on a cart 44 configured to be moved about the medical facility. A manifold receiver 46 is supported on the cart 44 and configured to removably receive implementations of a manifold 60-70 to be described in further detail. The manifold 60-70 is configured to receive a liquid tube coupler of a suction tube, after which a liquid flow path is established from a suction instrument to the waste canister 42 under the influence of a vacuum from the vacuum source. The liquid medical waste is collected within the waste canister 42, and filtered air from the vacuum source is discharged through an exhaust 56. Another implementation of the waste collection system 40 is shown in FIG. 2 in which a second waste canister 43 is supported on the cart 44 and in fluid communication with the vacuum source. A second manifold receiver 47 is configured to receive another manifold 60-70 to establish fluid communication between the manifold 60-70 and the second waste canister 43.

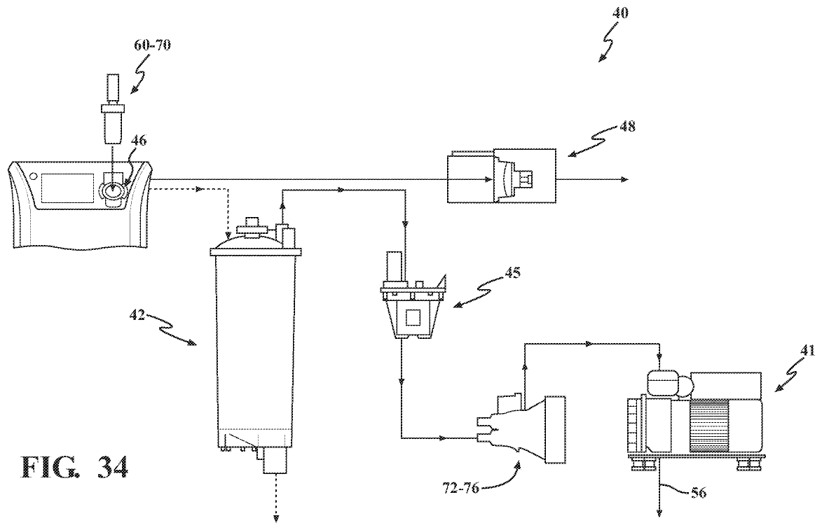

As to be described, there may be several types of manifolds 60-70 for varying purposes, e.g., only filtering liquid medical waste, only filtering surgical smoke, filtering liquid medical waste and surgical smoke). Thus, a first type of manifold 60-70 may be operably coupled with the first manifold receiver 46, and a second type of manifold 60-70 may be operably coupled with the second manifold receiver 47, thereby providing flexibility to attending medical staff to within and between surgical procedures that may or may not use electrocautery or other smoke-generating devices. Further, the waste collection system 40 may include a smoke evacuation unit 48 supported on the cart 34. The smoke evacuation unit 48 may include another vacuum source (see FIG. 34) configured to draw surgical smoke through additional suction lines separately coupled to the smoke evacuation unit 48. Certain subsystems of the illustrated implementations of the waste collection system 40 are disclosed in the aformentioned International Publication No. WO 2007/070570, International Publication No. WO 2014/066337, International Publication No. WO 2017/112684, and International Publication No. WO 2020/210763.

In certain implementations, the waste collection system 40 includes a filter receptacle 50 configured to removably and interchangeably receive implementations of a system filter 72-76 to be described in further detail. FIGS. 1 and 2 show the removable cover disposed on the filter receptacle 50. A data reader 52, 53 may be associated with each of the manifold receiver(s) 46, 47 and/or the filter receptacle 50. For example, the data reader 52 may be a radiofrequency (RFID) reader operably coupled to the manifold receiver(s) 46, 47, or another example includes the data reader 52 being a plug-in-socket connection disposed within the filter receptacle 50. The data readers 52, 53 are in electronic communication with a controller 54 supported on the cart 34. For reasons to be described, the data readers 52, 53 are configured to detect the manifold 60-70 and the system filter 72-76 being operably coupled to a respective one of the manifold receiver(s) 46, 47 and the filter receptacle 50, and receive data from a data storage means coupled thereto. The data is transmitted to the controller 54 including one or more processors. Determinations therefrom may be made by the controller 54 for controlling the vacuum source 41 and/or a vacuum regulator 45 (see FIGS. 33-35), and/or providing alerts or other information on a user interface 49 supported on the cart 34.

Instead of a mobile cart, aspects of the present disclosure may be implemented on a console including means for establishing fluid communication between one or more waste canisters (e.g., standalone canisters) and one or more vacuum sources (e.g., facility-integrated suction). For example, the console may include ports to receive tubing, one or more manifold receivers, and/or one or more filter receptacles. The console may be supported on a tabletop surface, wherein the console effectively functions as a hub or junction between the manifold(s), the system filter(s), and the vacuum source(s). It is contemplated that the console may also include an electrosurgical unit for operating an electrosurgical tool that generates the surgical smoke. The aformentioned implementations of the waste collection system 40, the console-based system, and the like, may be collectively referred to herein as a suction system.

Referring again to FIG. 1, the waste collection system 40 may not include independent means for smoke evacuation, or alternatively the attending staff may not wish to utilize the smoke evacuation unit 48 of the waste collection system 40 of FIG. 2. To address such shortcomings or instances, the implementations of the manifold 60-70 of the present disclosure advantageously provide for evacuation of surgical smoke through the manifold receiver(s) 46, 47 under influence of the vacuum from the vacuum source 41, and do so in a manner that comports with the filtration performance demanded for surgical smoke evacuation (e.g., regulatory or certification standards). In manners to be further described, ensuring the filtration performance is optimal or satisfied may be determined in an automatic manner by the controller 54, thereby preventing instances in which the attending medical staff is unknowingly exposed to excessive surgical smoke. Moreover, certain implementations of the manifold 60, 64, 66, 68 provide for evacuation of the liquid medical waste, selectively or simultaneously, with the surgical smoke, if desired.

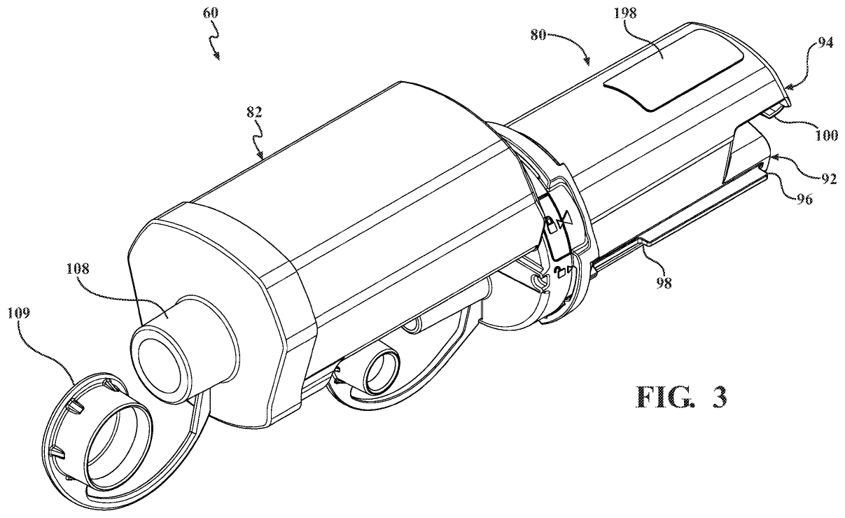

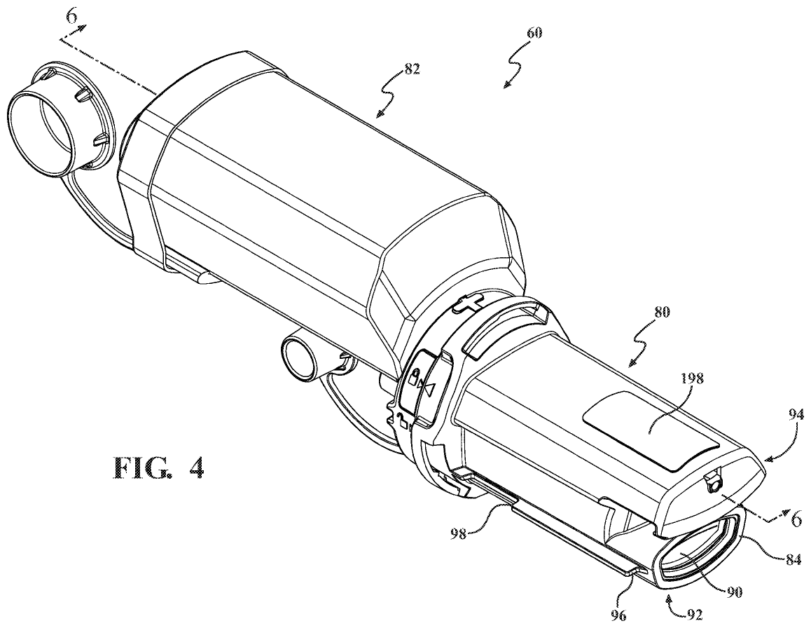

Referring now to FIGS. 3-6, a first implementation of the manifold 60 includes a trunk 80, and a head portion 82 coupled to the trunk 80. The trunk 80 defines an outlet opening 84 configured to be inserted into the manifold receiver(s) 46, 47 of the waste collection system 40. The trunk 80 further defines a body interior 86 in communication with the outlet opening 84. A filter basket 88 may be disposed within the body interior 86 of the trunk 80. The filter basket 88 is configured to filter solid and semi-solid material entrained within liquid being drawn into the manifold 60 from the suction instrument (e.g., a Yankeuer instrument). A seal 90 may be coupled to the trunk 80 to cover the outlet opening 84. The seal 90 is configured to be engaged by a complementary component of the manifold receiver(s) 46, 47 to provide a sealed liquid flow path through the manifold 60 and to the waste canister(s) 42, 43.

The trunk 80 includes features or geometries configured to engage complementary components within the manifold receiver(s) 46, 47. The features may include a first leg 92, a second leg 94 spaced apart from the first leg 92 to define a void, arms 96, lock elements 98, catches 100, and a spine 102. At least a portion of the arms 96 may be positioned on the first leg 92, and the lock elements 98 may be positioned distal to the arms 96. The catches 100 may be disposed on the second leg 94 and spaced apart from the arms 96 by the void. The spine 102 may extend an underside of the trunk 80 and include a ramped surface. The interaction between the aformentioned structures of and complementary structures of the manifold receiver(s) 46, 47 is disclosed in the aformentioned International Patent Publication No. 2020/209898. Alternatively, implementations of the manifold 60-70 disclosed herein may be an in-line device in which the housing is not insertable into a receiver but rather includes an inlet fitting and an outlet fitting each configured to receive suction tubes.

In certain implementations, the head portion 82 is removably coupled to the trunk 30. Each of the head portion 82 and the trunk 80 may include complementary coupling features configured to be selectively engaged and disengaged to permit access to the body interior 86 of the trunk 80, for example, through a rotational input. In particular, it may be desirable access the filter basket 88 to retrieve a polyp or other tissue sample that may have been suctioned into the manifold 60. Alternatively, the head portion 82 and the trunk 80 may be rigidly secured to one another through spin welding, laser welding, or the like, or otherwise the head portion 82 and the trunk 80 may be integrally formed as a monolithic structure. The trunk 80 and the head portion 82, taken singly or in combination, may be referred to herein as a housing of the manifold 60.

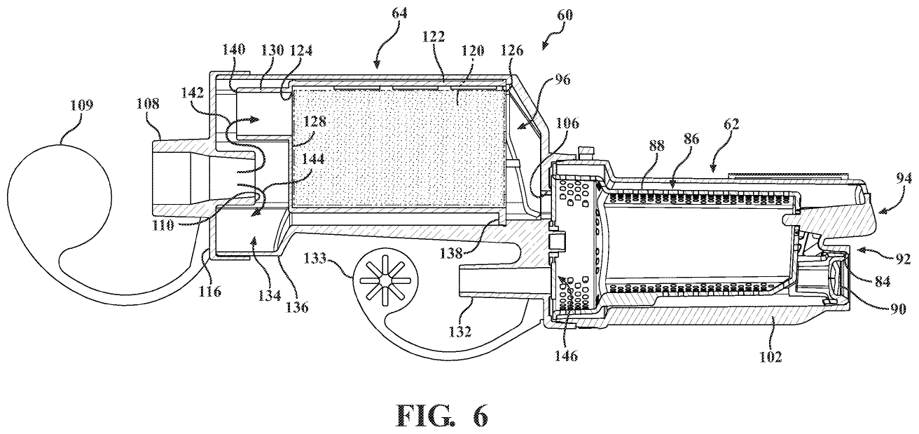

The head portion 82 defines a head interior 104, and an aperture 106 provides fluid communication between the head interior 104 and the body interior 86, as best shown in FIG. 6. The head portion 82 includes an inlet fitting 108 configured to be removably coupled with a smoke tube coupler of a smoke evacuation instrument. The inlet fitting 108 defines a bore to a proximal end 110 of the inlet fitting 108 that is positioned within the head interior 104. The bore of the inlet fitting 108 may include a proximal inner diameter, a distal inner diameter greater than the proximal inner diameter, and a ramped surface between the proximal and distal inner diameters. The proximal and distal inner diameters are sized to accommodate smoke tube couplers of different sizes. Should it be desired for the manifold 60 not be used for surgical smoke evacuation, a fitting cap 109 may be secured over the inlet fitting 108.

The head portion 82 including a proximal section 112 and a distal cap 114 secured to the proximal section 112. The proximal section 112 and the distal cap 114 may be integrally formed through blow molding, injection molding or the like, or alternatively formed separately and joined to one another through adhesive or another a suitable joining process. The distal cap 114 may include a cap faceplate 116 with the inlet fitting 108 extending from the cap faceplate 116. The cap faceplate 116 may define a distal boundary of the head interior 104.

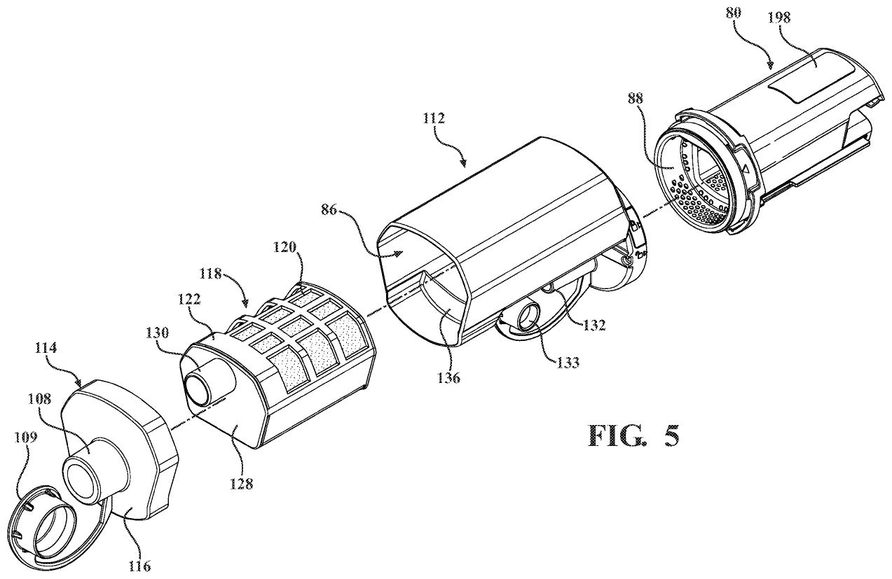

The manifold 60 includes a filter assembly 118 disposed within the head interior 104. The filter assembly 118 includes a particulate filter 120 configured to filter the surgical smoke being drawn smoke evacuation instrument through the inlet fitting 108. For example, the particulate filter 120 may be an ultra-low particulate air (ULPA) filter, a high-efficiency particulate air (HEPA) filter, a combination thereof, and/or other filtering layers and filtering media to be described. In certain implementations, the particulate filter 120 is disposed within a filter casing 122. As best shown in FIG. 6, the filter casing 122 defines an inlet 124 and an outlet 126 with the particulate filter 120 disposed between the inlet 124 and the outlet 126. With further reference to FIG. 5, the filter casing 122 may generally be box-shaped and contoured to confirm to inner surfaces of the head portion 82, and more particularly the proximal section 112 of the head portion 82. The filter casing 122 may assume other suitable geometries, and may not conform to the head portion 82. The filter casing 122 may be secured to the head portion 82 through any suitable joining means, including interference fit, adhesive, laser welding, or the like. The filter casing 122 may include a distal barrier 128, and a port 130 extending distally from the distal barrier 128. A proximal end of the port 130 may open through the distal barrier 128 to define the inlet 124 of the filter casing 122, and the port 130 further defines a bore in fluid communication therewith. It is appreciated that in certain variants the port 130 is optional, and the distal barrier 128 may define the inlet 124 with no structure extending distally therefrom. An upper aspect of the filter casing 122 may define one or more openings (not identified) to facilitate gas is being drawn into the particulate filter 120. It is further contemplated that the filter casing 122 is optional, and the particulate filter 120 may be secured directly to inner surfaces of the head portion 82 to provide a sealed pathway therethrough. For example, the particulate filter 120 may be joined to or supported by the inner surfaces with an adhesive or a gasket.

In certain implementations, the head portion 82 includes a bypass fitting 132 configured to receive the liquid tube coupler of a liquid suction instrument (i.e., distinct from the smoke evacuation instrument). The bypass fitting 132 is in fluid communication with the body interior 86. In particular, the liquid medical waste being drawn through the suction instrument and through bypass fitting 132 bypasses the filter assembly 118 to be drawn directly into the body interior 86. The arrangement advantageously provides for evacuation of liquid medical waste without compromising the particulate filter 120. In other words, the surgical smoke being drawn through the manifold 60 is filtered with the particulate filter 120, and the liquid medical waste being drawn through the manifold 60 is filtered with the filter basket 88, perhaps simultaneously. In instances where the manifold 60 not being used for liquid waste evacuation, another fitting cap 133 may be secured over the inlet fitting 132.

As mentioned, there may be trace liquid medical waste that is suctioned together with the surgical smoke. Excessive amounts of the liquid medical waste may compromise the effectiveness of the particulate filter 120. The manifold 60 of the present disclosure addresses the aforementioned technical challenge by including a fluid sump 134 and means for separating the liquid medical waste from the surgical smoke. With continued reference to FIGS. 5 and 6, a distal length of the head portion 82 may include a lower wall or protrusion 136 that flares downwardly from an adjacent proximal length of the lower wall. A subvolume of the head interior 104 defined by the protrusion 136 may be considered the fluid sump 134. The fluid sump 134 may be at least partially disposed below the filter casing 122 (or the particulate filter 120 in variants in which there is no filter casing 122). Further, the fluid sump 134 may be positioned between the distal barrier 128 of the filter casing 122 and the cap faceplate 116 of the head portion 82. As best shown in FIG. 6, the filter casing 122 may include a seal 138 separating the fluid sump 134 from a region of the head interior 104 proximal to the filter assembly 118. For example, the seal 138 may be a gasket, or the illustrated implementation of the seal 138 is a flange of the filter casing 122 engaging an inner surface of the head portion 82. In such an implementation, the fluid sump 134 may extend beneath the filter casing 122 between the cap faceplate 116 and the proximal flange 138. In other words, the fluid sump 134 includes a gap defined between the filter casing 122 and a lower wall of the head portion 82. The volume of liquid medical waste of which the fluid sump 134 may be able to accommodate at least 1 milliliter (mL), at least 5 mL, or at least 10 mL or more of liquid.

To facilitate separation of the trace liquid from the surgical smoke, the inlet fitting 108 and the port 130 of the filter casing 122 may be in a staggered arrangement. In particular, the proximal end 110 of the inlet fitting 108 is positioned proximal to a distal end 140 of the port 130 of the filter casing 122. Further, the distal end 140 of the port 130 may be positioned above the proximal end 110 of the inlet fitting 108. As a result, fluid entering the manifold 60 is effectively separated into a gas flow path (arrow 142), and a liquid flow path (arrow 144) that descends into the fluid sump 134 under the influence of gravity. In other variants, the inlet fitting 108 and the port 130 may be offset in any suitable direction and orientation. For example, the inlet fitting 108 and the port 130 may be laterally offset to one another, and/or the distal end 140 of the port 130 may be positioned below the proximal end 110 of the inlet fitting 108. Based on the staggered arrangement of the inlet fitting 108 and the port 130, the tortuous path prevents at least most of the trace liquid from being drawn through the inlet 124 of the filter assembly 118.

In the implementation of FIG. 6, the liquid medical waste is drawn through the bypass fitting 132 and directly into the body interior 86, as mentioned, defining a liquid flow path in the trunk 80. The gas flow path is configured to pass through the inlet 124, through the particulate filter 120, and through the aperture 106 to merge or join the liquid flow path within the trunk 80. The combined fluid flow paths (i.e., the filtered surgical smoke and the liquid medical waste) are drawn through the outlet opening 84. A backflow prevention valve 146 may be disposed within the body interior 86 and configured to prevent backflow from the liquid flow path to the gas flow path. In particular, the backflow prevention valve 146 may include resilient flappers selectively covering the aperture 106, and a proximal end of the bypass fitting 132.

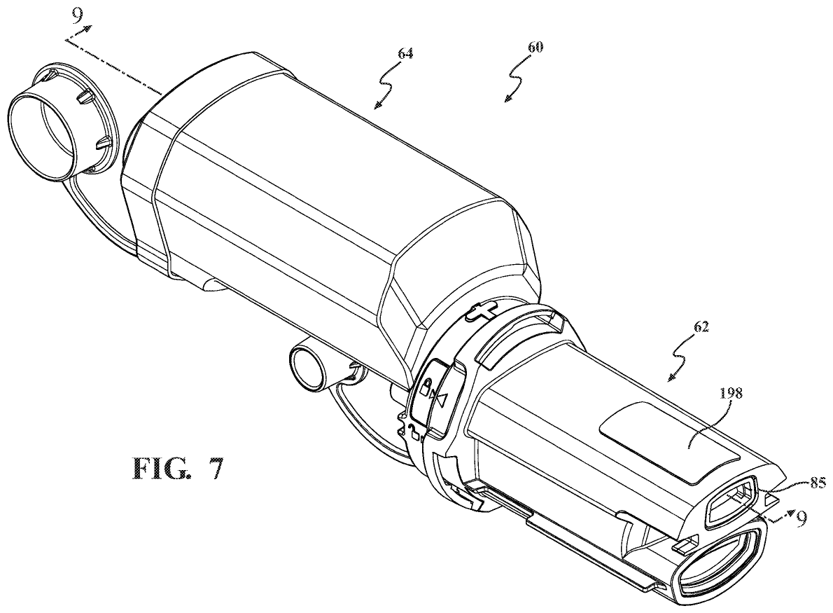

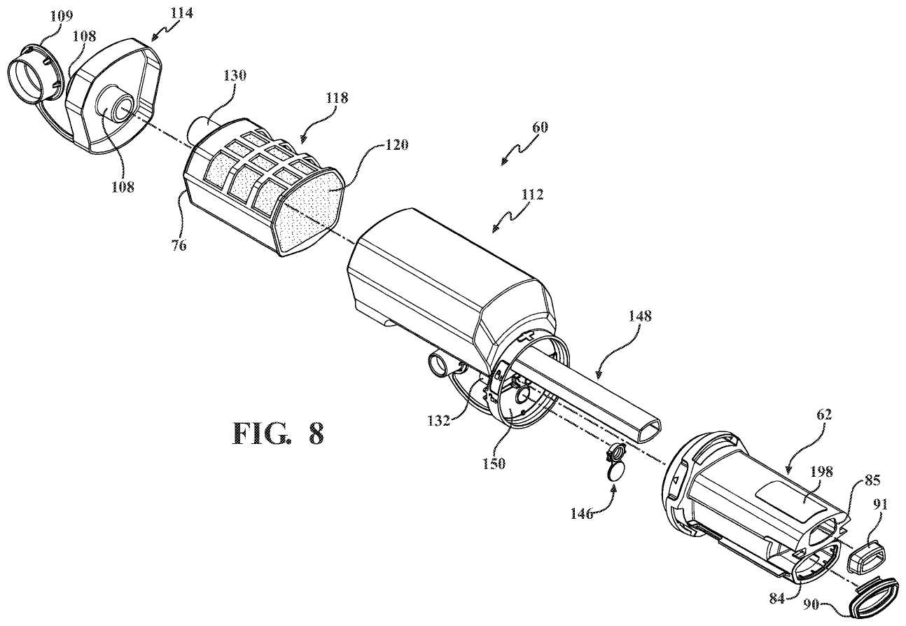

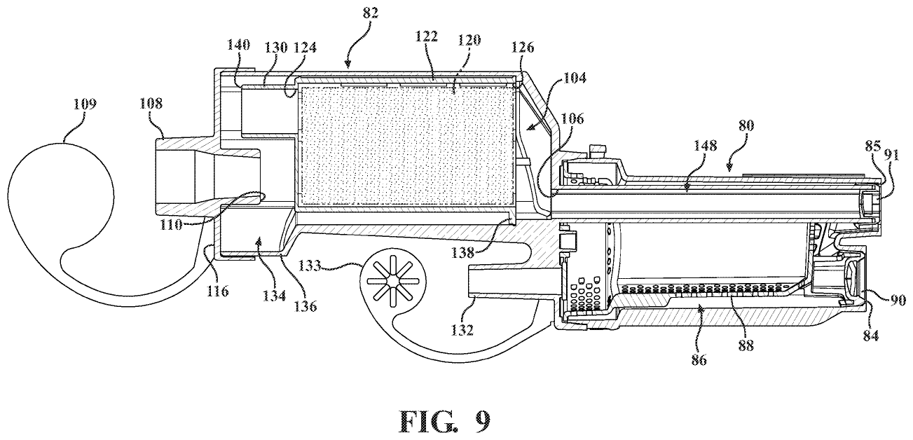

FIGS. 7-9 illustrates a variant of the manifold 60 with like numerals indicating like components, wherein a second seal 91 is disposed within a second outlet opening 85. The manifold 60 includes a conduit 148 configured to prevent fluid communication between the gas flow path and the liquid flow path. In particular, and with reference to the sectional view of FIG. 9, the conduit 148 is coupled to the head portion 82, and more particularly to the proximal section 112 of the head portion 82. Even more particularly, the head portion 82 may include a neck section 150 configured to be directly coupled to the trunk 80 with the proximal section 112 extending distally from the neck section 150. The conduit 148 may include a distal end engaging the neck section 150 and positioned to align with the aperture 106. The conduit 148 extends proximally from the neck section 150, and opposite the distal end, the conduit 148 may include a proximal end in fluid communication with the second outlet opening 85. The proximal end of the conduit 148 may be positioned to support the second seal 91 within the second outlet opening 85, as best shown in FIG. 9.

The arrangement results in the gas flow path passing through the casing inlet 124, through the particulate filter 120, and through the aperture 106 to further pass through the conduit 148 and the second outlet opening 85. The liquid flow path passing through the body interior 86 remains fluidly separated from the gas flow path to pass through the first outlet opening 84. It is understood that corresponding modifications to the manifold receiver(s) 46, 47 may be necessitated to provide for the separate gas flow path through the waste collection system 40 (see FIG. 34).

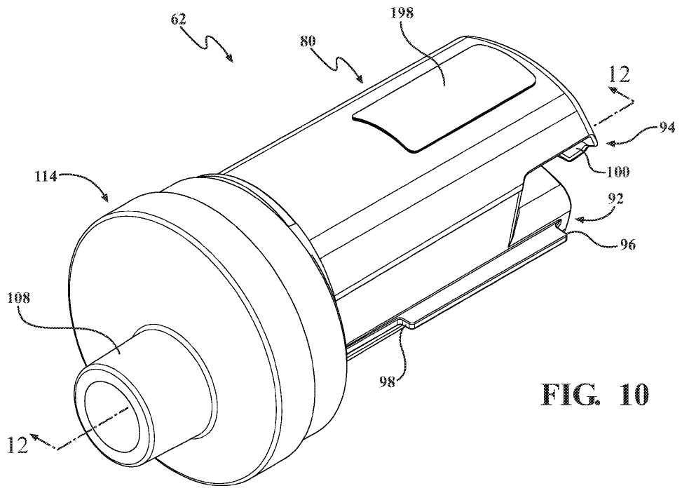

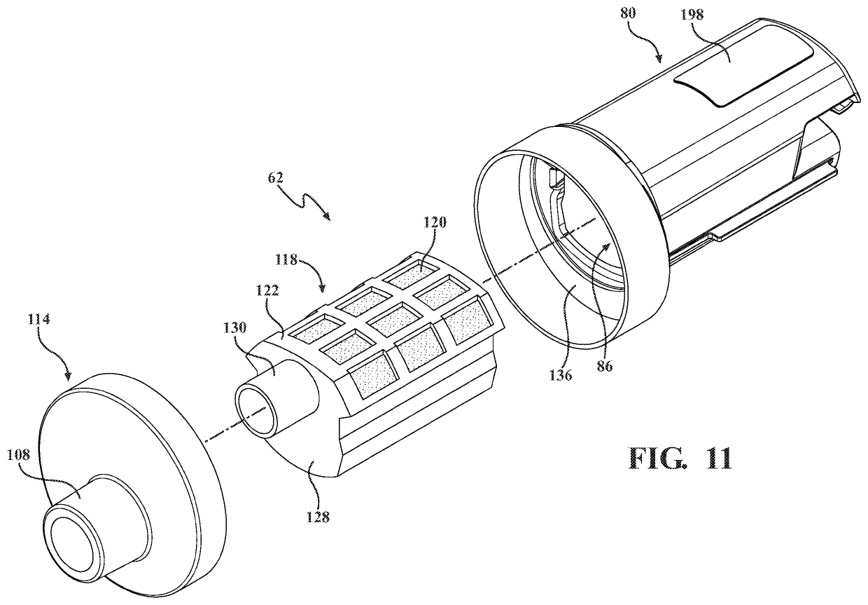

Another implementation of the manifold 62 is shown in FIGS. 10-12 with like numerals indicating like components, wherein separate evacuation of liquid medical waste is not provided. In particular, the manifold 62 does not include the bypass fitting 132 for bypassing the filter assembly 118. Likewise, in any of the implementations disclosed herein, the bypass fitting 132 is optional and the manifold 60-70 may be designed solely for evacuation of surgical smoke.

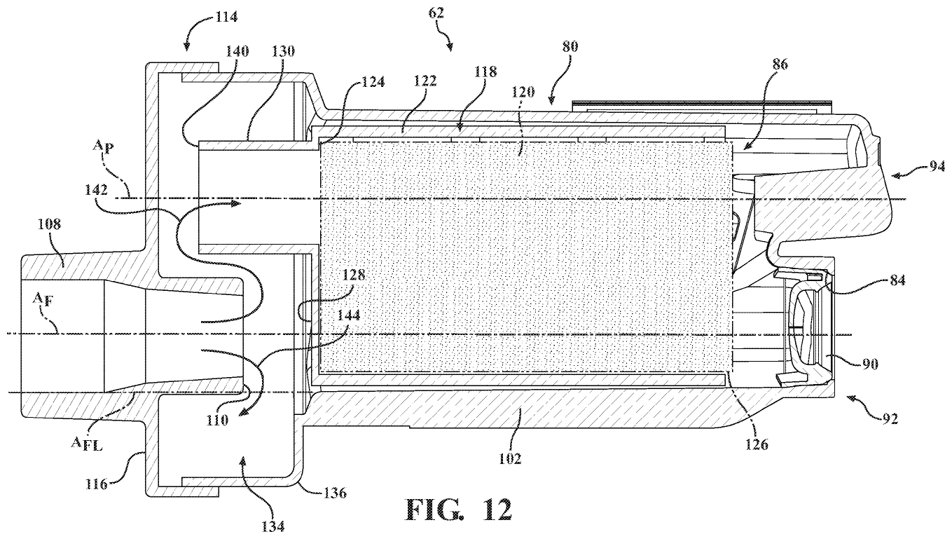

The manifold 62 includes the filter assembly 118 positioned within the trunk 80. The head portion 82 of the illustrated embodiment may be considered akin to the distal cap 114 of the previous embodiment. With reference to FIG. 12, the fluid sump 134 is at least partially disposed below (in elevation) the first leg 92 of the trunk 80. In particular, an axis (A_{FL}) or plane of a lower inner surface of the first leg 92 that defines the outlet opening 84, extended distally, is above a bottom of the fluid sump 134. Further, an axis (A_{P}) of the port 130 of the filter casing 122 is at least substantially aligned (in elevation) with the second leg 94. Still further, an axis (A_{F}) of the inlet fitting 108 is at least substantially aligned (in elevation) with the first leg 92. Further, as previously described, the inlet fitting 108 and the port 130 are in the staggered arrangement to separate the fluid into the gas flow path (arrow 142), and the liquid flow path (arrow 144) to descend into the fluid sump 134 under the influence of gravity.

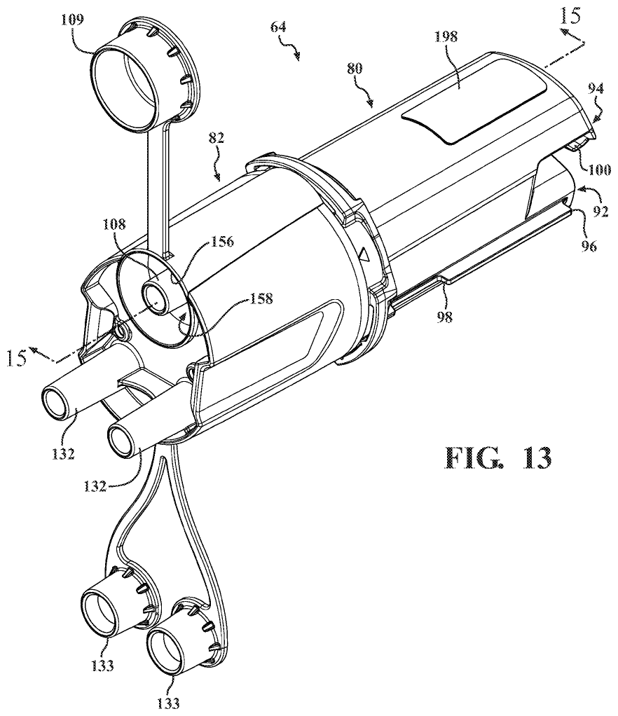

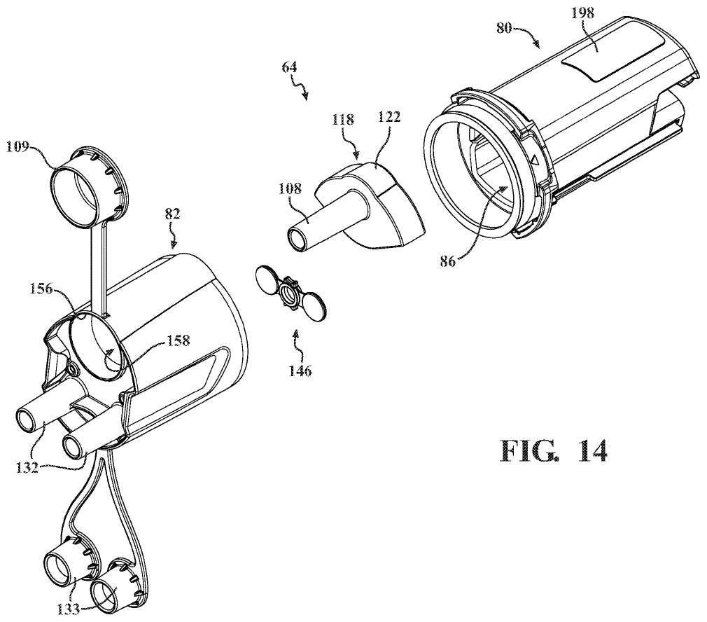

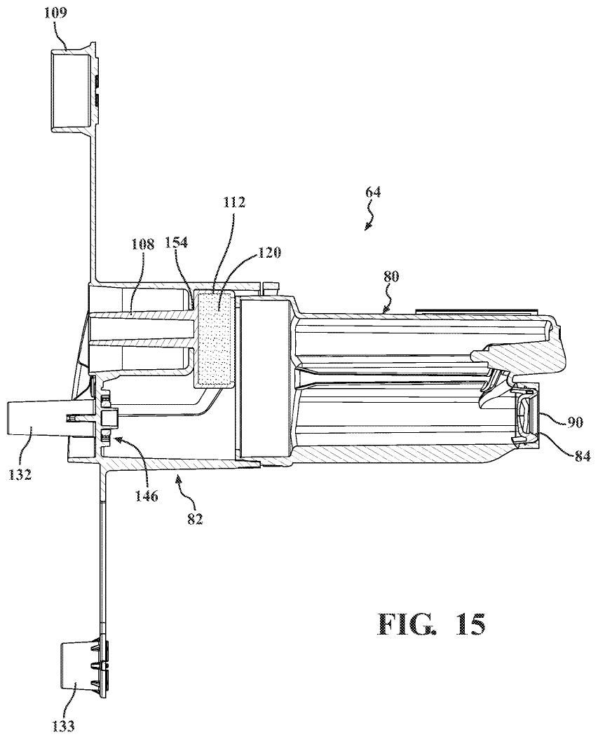

Referring to another implementation of the manifold 64 shown in FIGS. 13-16, with like numerals indicating like components, the head portion 82 may define a bore 154, and the filter assembly 118 includes the inlet fitting 108 extending through the bore 154 of the head portion 82. In particular, the filter casing 122 is integrally formed with the inlet fitting 108 to provide for direct connection between the smoke tube coupler and the filter assembly 118. In certain implementations, the head portion 82 includes a rim 156 defining a cavity 158, wherein the bore 154 is defined at a base of the cavity 158. The rim 156 and the inlet fitting 108 may be concentrically arranged. The cavity 158 may be sized to a known size and/or shape of conventional smoke tube couplers to provide further securing means and/or to avoid user confusion between the inlet fitting 108 and the bypass fitting(s) 132. The diameter of the rim 156 may be less than the diameter of typical liquid tube couplers, but greater than or equal to the diameter of typical couplers for smoke suction tubes. Further visual assistance may be provided by the head portion 82 being translucent or transparent, and the filter casing 122 is formed from colored material corresponding to a color associated with the coupler of the smoke suction tube. In a non-limiting example, the filter casing 122 is green and compatible couplers of smoke suction tubes are green. The filter casing 122 may be secured to the head portion 82 through any suitable joining means, including interference fit, adhesive, laser welding, or the like. The particulate filter 120 is disposed within the filter casing 122.

The bypass fittings 132 are in fluid communication with the body interior 86. The liquid medical waste being drawn through the suction instrument and through bypass fittings 132 bypasses the filter assembly 118 to be drawn directly into the body interior 86. Again, in instances where only one of the suction instrument and the smoke evacuation instrument is being utilized, the other one of the inlet and bypass fittings 108, 132 may be closed with a respective one of fitting caps 109, 133 coupled to the head portion 82. The backflow prevention valve 146 may be coupled to an inner surface of the head portion 82 and sized to resiliently cover inner opening(s) of the one or more bypass fittings 132.

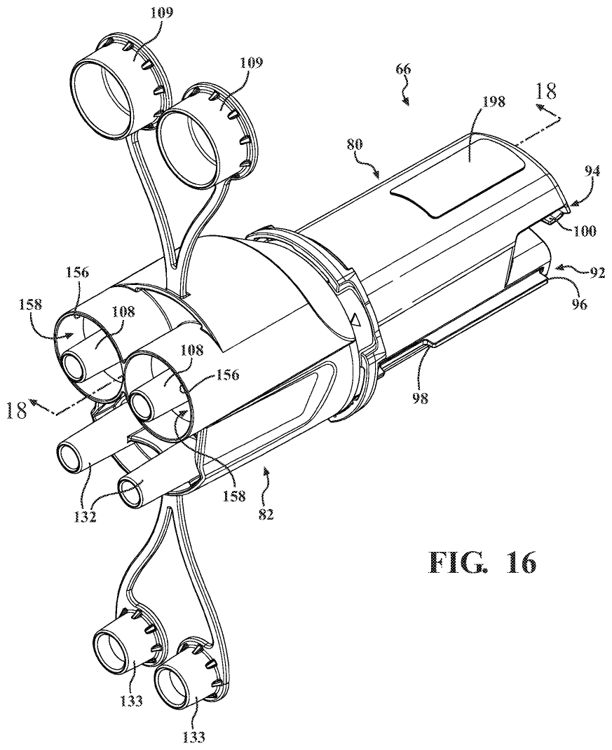

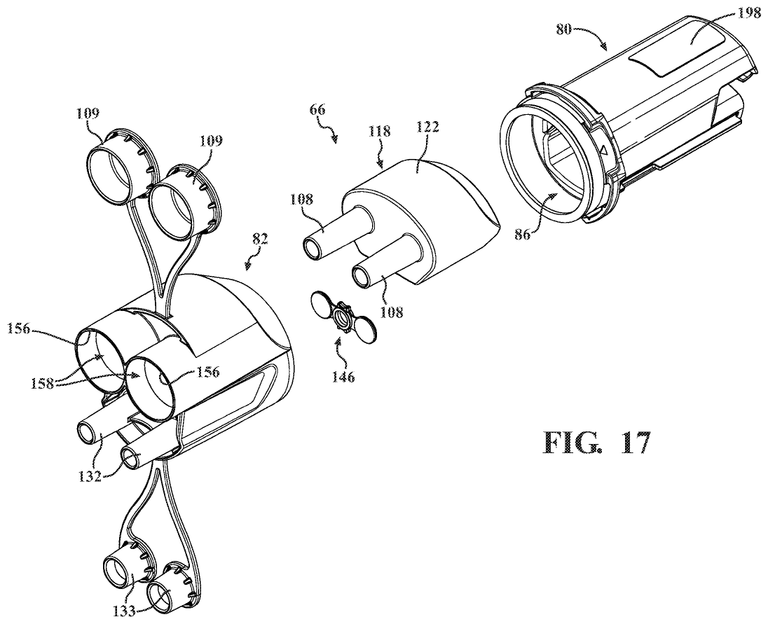

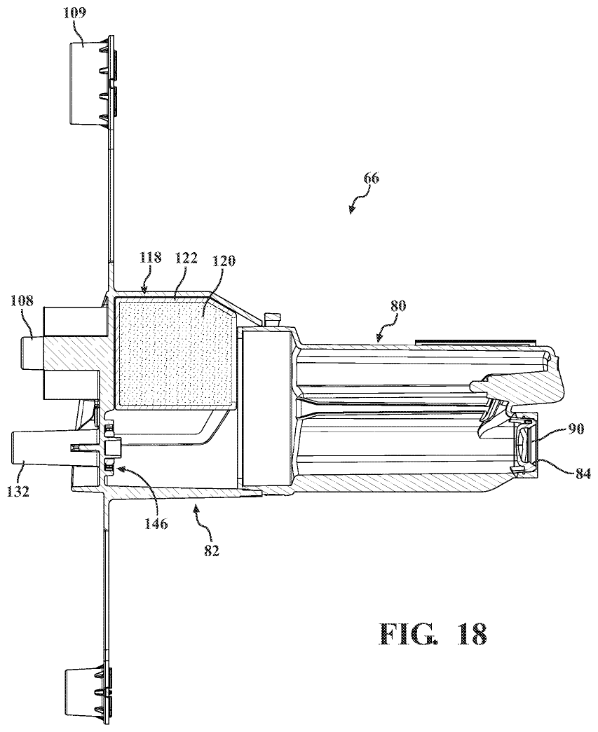

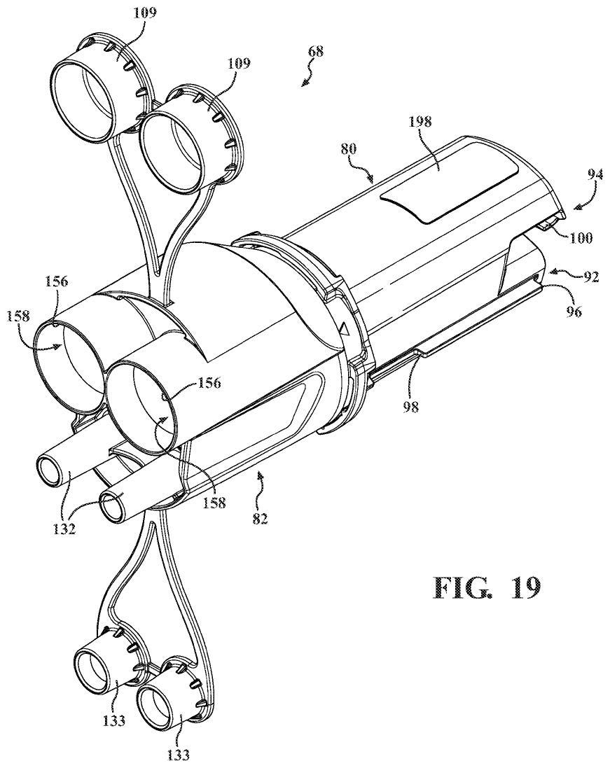

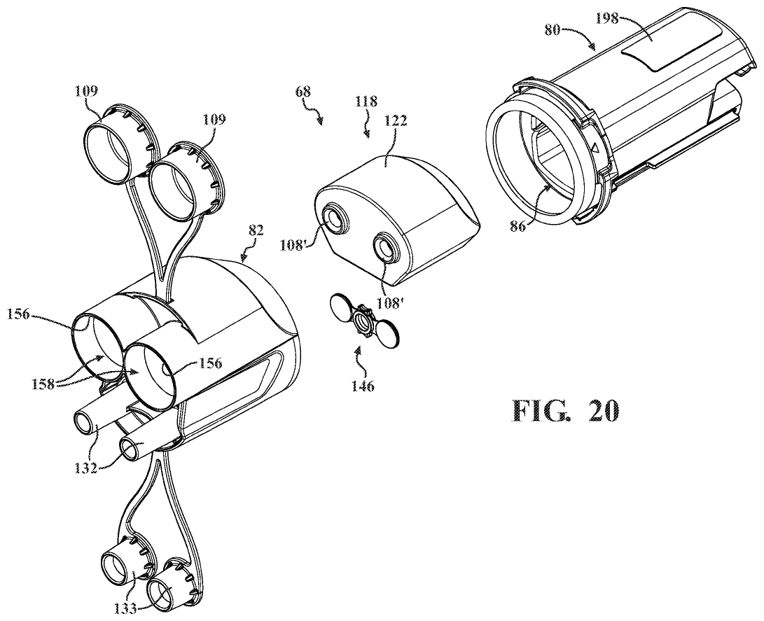

FIGS. 16-18 depict a variant of the manifold 66 in which there are two inlet fittings 108 each configured to receive one of two smoke tube couplers. The rim 156 surrounding the inlet fittings 108 may contact one another such that the inlet fittings 108 are in a side-by-side arrangement. The filter casing 122 and a particulate filter 120 disposed therein may be relatively larger in size to accommodate the potential for twice the flow of surgical smoke being drawn into the manifold 66. FIGS. 19 and 20 depict still another variant of the manifold 68 in which there are inlet ports 108′ as opposed to inlet fittings extending within the cavity defined by the rim(s) 156. The inlet ports 108′ may be sized and shaped to receive a known configuration of smoke tube couplers.

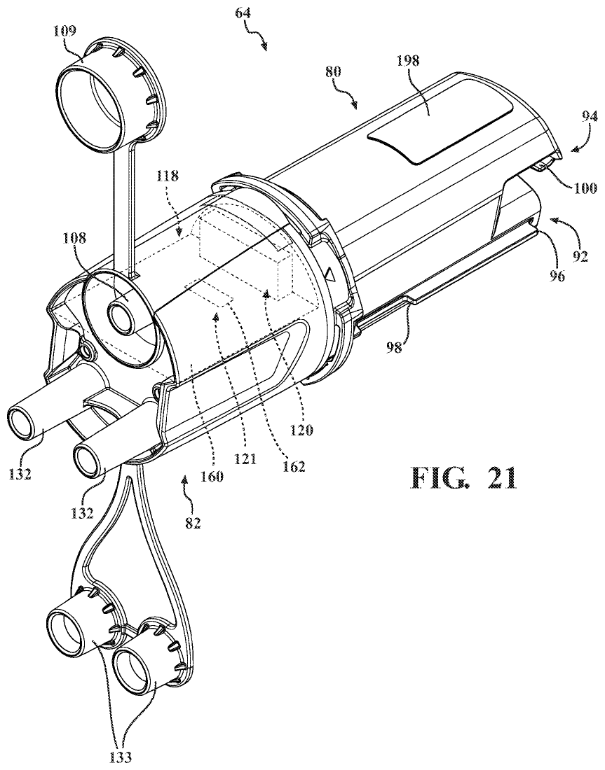

Referring now to FIGS. 21-26, certain optional features of the filter assembly 118 are depicted. The features are illustrated on the third implementation of the manifold 64; however, the features may be included on any of the implementation depicted and described herein. FIG. 21 shows the head portion 82 including a barrier 160. The barrier 160 may define a window or aperture 162 and otherwise be impermeable. For example, the barrier 160 may be formed integrally with the material forming the head portion 82. The filter assembly 118 includes the particulate filter 120, hereinafter referred to as a primary or first particulate filter 120. The illustrated implementation shows the first particulate filter 120 supported on the barrier 160, but alternatively may be supported above the barrier 160. It is further contemplated that the first particulate filter 120 may be disposed within a filter casing, for example, any of the implementations previously described.

The barrier 160 may define the aforementioned fluid sump 134 such that trace liquid within the surgical smoke is collected thereon. The filter assembly 118 includes a supplemental or second filter media 121 disposed within the head portion 82, and more particularly disposed within the aperture 162. The first particulate filter 120 may be formed from media configured to cause a low pressure drop of the surgical smoke (e.g., less than 5%) being drawn therethrough. The second filter media 121 may be formed to cause a higher pressure drop to prevent excessive amounts of gas from being drawn therethrough, yet permit the trace liquid to seep therethrough to join the liquid flow path, e.g., being drawn through the bypass fitting(s) 132. In one non-limiting example, the first particulate filter 120 is pleated, whereas the second filter media is mixed media. Thus, the first particulate filter 120 and the second filter media 121 are formed with different filtering characteristics. Again, the filtering characteristics of the first particulate filter 120 may be to filter smoke particulates, and the filtering characteristics of the second filter media 121 may be to filter liquid and/or liquid and smoke particulates.

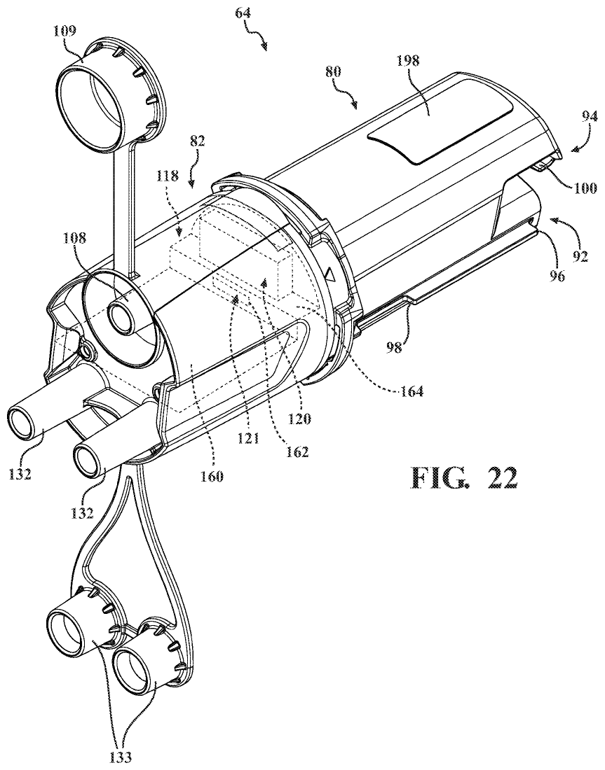

FIG. 21 shows the second filter media 121 generally located centrally within the barrier 160, but any suitable location is contemplated, and more than one second filter media 121 may be provided. FIG. 22 depicts a variant in which the barrier 160 includes a step 164, and the aperture 162 is defined in a distal face of the step 164. The first particulate filter 120 is supported on the step 164, and the second filter media 121 is disposed within the aperture 162 on the distal face. Thus, the first particulate filter 120 is supported above the fluid sump 134 defined by the barrier 160.

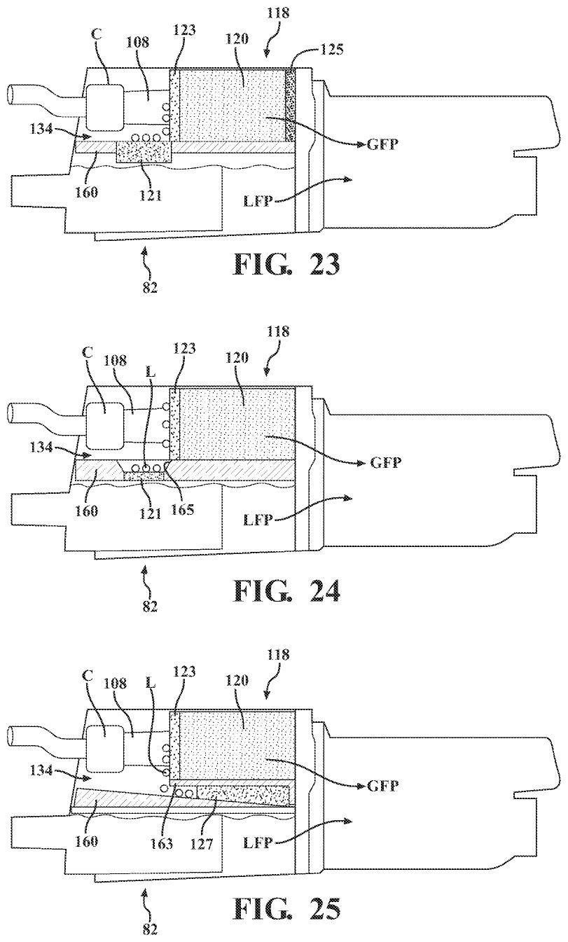

Referring now to FIGS. 23-25, the filter assembly 118 may include means for separating the liquid medical waste from the surgical smoke. One variant includes a prefilter layer or media 123 coupled to or positioned upstream of the first particulate filter 120. The prefilter media 123 may be treated or formed with a coalescing material, in particular a coating that is repellant to liquids, such as a hydrophobic or oleophobic coating, a coating of per- and polyfluoroalkyl substances (PFAS), or the like. Upon contact with the prefilter media 123, the liquid is promoted to agglomerate or coalesce on or within the prefilter media 123 rather than pass therethrough. The gas of the surgical smoke passes through the prefilter media 123 and into the primary particulate filter 120 to form the gas flow path (GFP). As generally shown, the denser liquid descends along an outer surface of the prefilter media 123 into the fluid sump 134 under the influence of gravity. The liquid collects within the fluid sump 134, and weeps or seeps into the liquid flow path (LFP) through the second filter media 121. In the variant depicted in FIG. 24, the barrier 160 includes a cavity 165 within which the second filter media 121 is supported, thereby promoting the liquid to collect on the second filter media 121 and promote seepage therethrough.

FIG. 25 depicts the barrier 160 including a ramped surface or otherwise being angled. The first particulate filter 120 (and the prefilter media 123) is supported on an upper partition 163 spaced above the barrier 160 to form a liquid collection compartment within the head portion 82. A sorbent 127 may be disposed within the liquid collection compartment. The sorbent 127 may be one or more pieces of foam media or super-absorbing fiber or polymer to limit or eliminate sloshing of liquid therein. The sorbent 127 may be one or more flat pads sized to the dimensions of the liquid collection compartment. The sorbent may include an antimicrobial agent to limit or prevent odors, and/or a reagent configured to change color when saturated with liquid.

The filter assembly 118 of any of the implementations disclosed herein may include at least one optional adsorber 125. FIG. 23 shows the adsorber 125 as a layer or media disposed downstream of the first particulate filter 120 (and the prefilter media 123). In a non-limiting example, the adsorber 125 is a charcoal filter or catalyst to remove odors from gaseous pollutants such as volatile organic compounds or ozone.

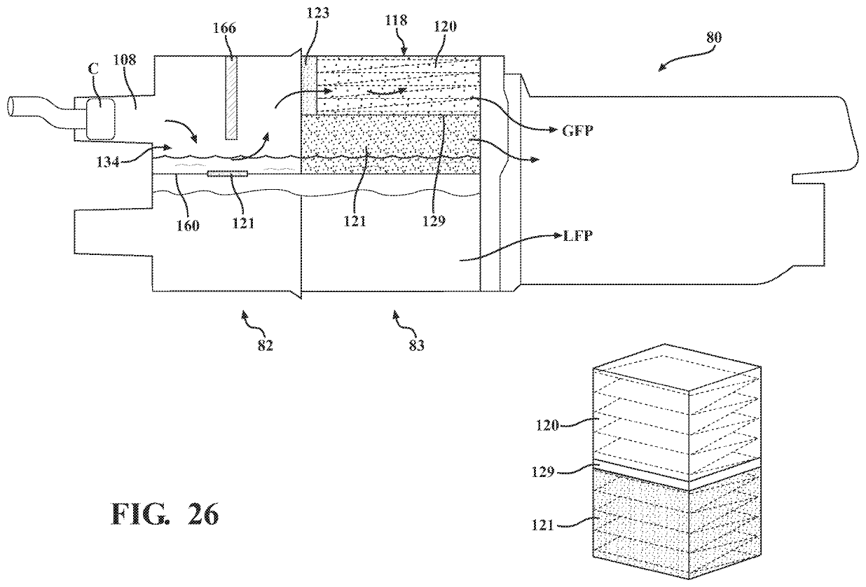

Referring now to FIG. 26, another implementation of the filter assembly 118 depicts additional optional features of the head portion 82. A baffle 166 extends downwardly from an upper interior surface of the head portion 82 and positioned between the inlet fitting 108 and the filter assembly 118. The baffle 166 is configured to separate the liquid from the surgical smoke for the liquid to be collected with the fluid sump 134. In other words, the surgical smoke, including liquid and gas, may encounter the baffle 166, after which the liquid is drawn downwardly along the baffle 166, after which momentum of the fluid about a lower edge of the barrier 192 causes the liquid to be further removed from the gas.

Further, the first particulate filter 120 and the second filter media 121 may be coupled to or otherwise separated by an impermeable separator 129. The first particulate filter 120 may be disposed above the separator 129, and the second filter media 121 maybe disposed the separator 129. The separator 129 may be oriented horizontally as shown, or the separator 129 may be oriented downwardly in a proximal direction. The illustrated implementation includes the first particulate filter 120 and the second filter media 121 being stacked vertically, however, other suitable arrangements are contemplated. For example, the first particulate filter 120 and the second filter media 121 may be arranged in a side-by-side configuration. Further, the primary particulate filter 120 may be angled relative to vertical, and in particular downward in the proximal direction so as to maximize proportions of the first particulate filter 120 relative to the second filter media 121. For example, it may be desirable for the fluid sump 134 to be limited to 10 milliliters of liquid or less. In such an arrangement, the prefilter media 123 may be disposed on multiple faces of the first particulate filter 120. The arrangement may also obviate the need for a baffle.

The variant of FIG. 26 may include a three-piece construction; i.e., the trunk 80, the head portion 82, and an adapter portion 83 coupled to disposed between the trunk 80 and the head portion 82. In particular, owing to the geometries configured to support the primary particulate filter 120 (and the baffle 166), manufacturing efficiencies may dictate the geometries being formed on the adapter portion 83, after which the trunk 80 and the head portion 82 are coupled to opposing sides of the adapter portion 83 through a suitable joining process such as spin welding, laser welding, adhesive, fasteners, or removable coupling.

The prefilter media 123 may extend between the separator 129 and the upper aspect of the head portion 82. In other words, the prefilter media 123 may be disposed upstream of the first particulate filter 120, but not upstream of the second filter media 121. The exemplary implementation shows first particulate filter 120 as pleated media, and the second filter media 121 as mixed media including pleated media and foam. Optionally, another second filter media 121, hereafter referred to as a third filter media, may be disposed within the aperture defined by the barrier 160 as previously described. The prefilter media 123, the pleated media, and the mixed media may be provided on any of the implementations of the manifold 60-70 disclosed herein.

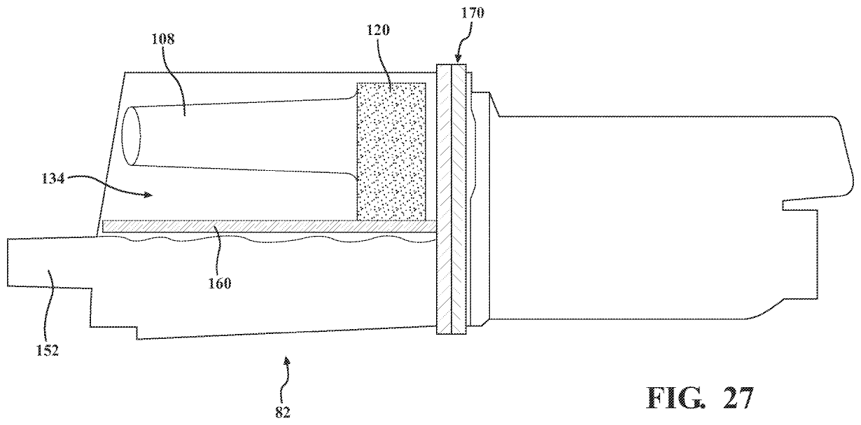

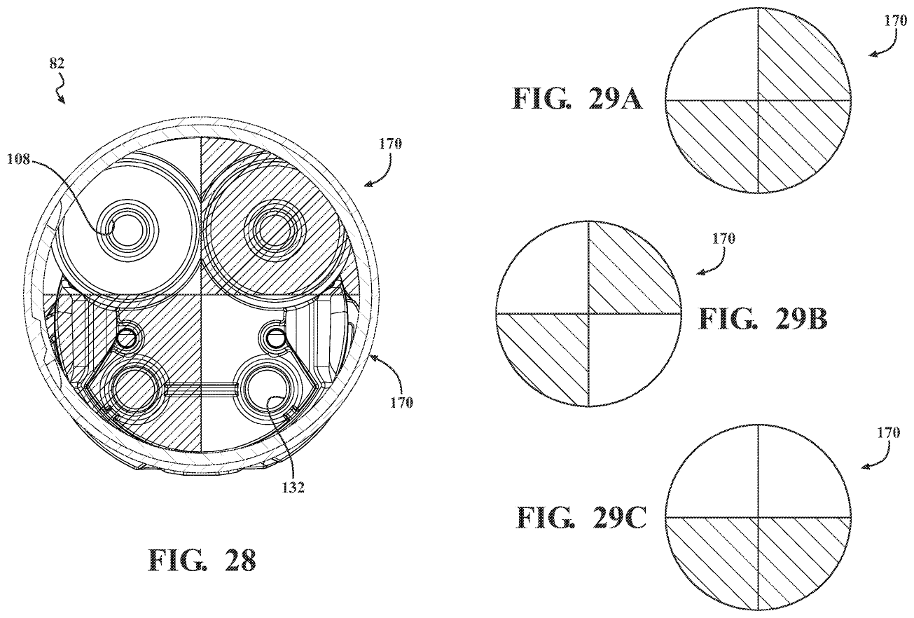

As mentioned, in instances where only one of the suction instrument and the smoke evacuation instrument is being utilized, the other one of the inlet and bypass fittings 108, 132 may be closed with a respective one of fitting caps 109, 133 coupled to the head portion 82. Additionally or alternatively, optional implementations of the manifold 60-70 include a valve assembly 170 configured to be actuated to move the manifold between fluid collection modes, namely a smoke collection mode, a liquid collection mode, or a combination thereof. A first variant of the valve assembly 170 is shown in FIGS. 27-29C in which one or more barriers configured to be rotated relative one another to permit or block fluid communication between a corresponding one of the inlet and bypass fittings 108, 132 and the outlet opening 84. The smoke collection mode may include permitting surgical smoke entering through one or both of the inlet fittings 108 to pass through the manifold (see FIGS. 29A and 29C). A dual collection mode may include permitting surgical smoke entering through one (or both) of the inlet fittings 108 as well as permitting liquid entering through one (or both) of the bypass fittings 132 to pass through the manifold (see FIGS. 28 and 29B). Alternative configurations to those depicted are contemplated.

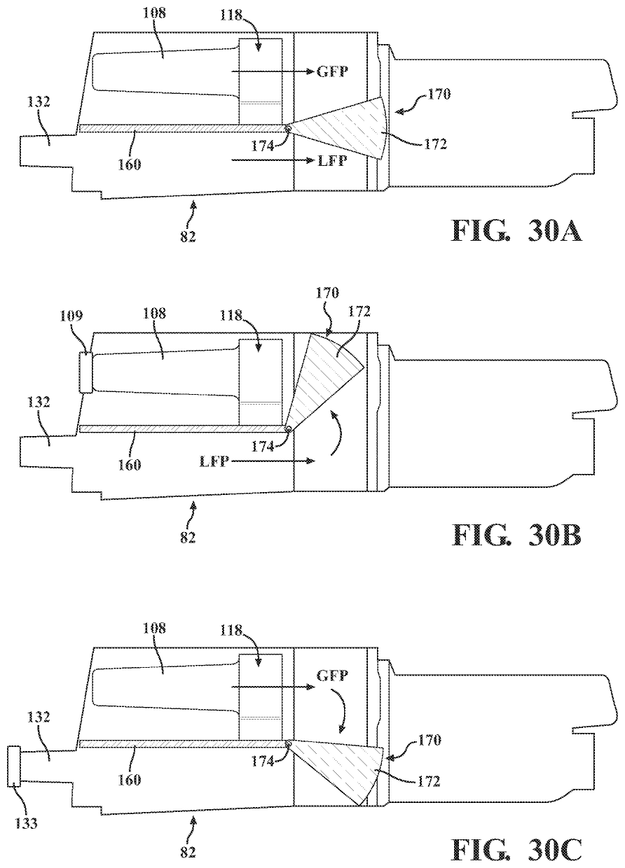

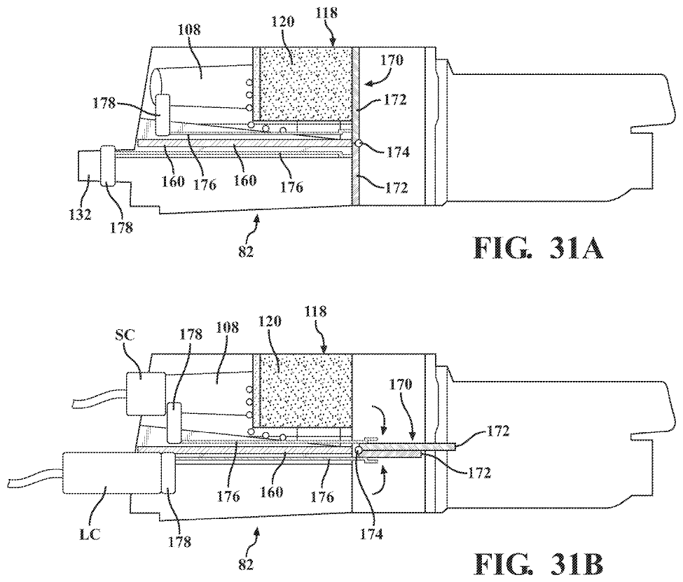

Variants of the valve assembly 170 includes a pivot valve depicted in FIGS. 30A-30C, and dual pivot valve depicted in FIGS. 31A and 31B. In a non-limiting example, the valve assembly 170 including a valving member 172 pivotably supported within the manifold 64, for example, at a pivot 174 coupled to the barrier 160 separating the gas flow path (GFP) from a liquid flow path (LFP). The valving member 172 may be biased (e.g., spring biased) to a neutral position shown in FIG. 30A, or alternatively, the valving member 172 may be unbiased so as to be pivotable without resistance. Based on whether the fitting cap 109 is coupled to the inlet fitting 108 or the fitting cap 133 is coupled to the bypass fitting 132, the valving member 172 is shaped to as to encounter the gas flow path and the liquid flow path. Based on relative strength between the gas flow path and the liquid flow path, the valving member 172 pivots accordingly. For example, should the fitting cap 109 be installed to block vacuum being drawn on the inlet fitting 108 (FIG. 30B) or the bypass fitting 132 (FIG. 30C), a lower pressure region distal to the valve assembly 170 causes the valving member 172 to pivot as shown.

The butterfly or dual pivot valve of FIGS. 31A and 31B includes two valving members 172 each supported within the manifold, for example, coupled to the barrier 160 at the pivot 174. One or more poppets include a strut 176, and a poppet head 178 operably coupled to or positioned adjacent to the inlet fitting 108 and the bypass fitting 132. The poppets are configured actuate the valving members 172 with coupling of the smoke tube coupler (SC) from the smoke suction tube and/or the liquid tube coupler (LC). The valving members 172 and/or the poppets may be biased the valve assembly 170 to a closed position shown in FIG. 31A in which the gas flow path and the liquid flow path are prevented. The closed position be associated with the cap(s) being coupled to the inlet or bypass fittings 108, 132, or no smoke or liquid coupler coupled thereto. FIG. 31B depicts the couplers from the smoke suction tube and the liquid suction tube being coupled to the inlet and bypass fittings 108, 132, from which the poppets are actuated to cause the valving members 172 to pivot to an open position of the valve assembly 170 in which the gas flow path and the liquid flow path are created for the surgical smoke and the liquid waste, respectively, to pass through the manifold 64. The valving members 172 and the poppets and independently deployable. Upon decoupling of the coupler(s), the valve assembly 170 returns to the closed position. An additional variant of the valving members 172 includes pinch valves configured to be actuated by actuators disposed on the system 40.

As depicted throughout the figures, any of the implementations of the manifold 60-70 may include means for storing manifold identification data. In one example, the means is an identification tag 198 coupled to the manifold 60-70, such as a radiofrequency identification (RFID) tag, an optical tag, or the like. The identification tag 198 may be coupled to the trunk 80 of the manifold 60-70. The identification data may include a presence or absence of the particulate filter 120, a presence or absence of the adsorber 125, presence or absence of the bypass fitting 132 (i.e., for use with liquid waste evacuation), or the like. Likewise, components and/or filtering characteristics of the implementations of the system filter 72-76 may be different, and the system filter 72-76 may also include means for storing system filter identification data. The means may include an identification tag, or memory storing data transmitted through a plug-in-socket connection, or the like. The identification data may include at least one of a system filter type, system filter filtration rating, presence or absence of a system filter adsorber, and the like. In a non-limiting example, a first system filter 72 may be an ultra-low particulate air (ULPA) filter, a second system filter 74 may be a high efficiency particulate air (HEPA) filter with an adsorber configured to remove volatile organic compounds (VOCs) or other pollutants (hereinafter “HEPA+”), and a third system filter 76 may be a HEPA filter. The implementations of the system filter 72-76 are interchangeably couplable with the filter receptacle 50 and may be selected based on the intended use of the system 40.

The data readers 52, 53 are configured detect the manifold 60-70 and the system filter 72-76 being operably coupled to a respective one of the manifold receiver(s) 46, 47 and the filter receptacle 50, and receive the identification data from a data storage means coupled thereto. The data is transmitted to the controller 54. Determinations therefrom may be made by the controller 54 for controlling the vacuum source 41 and/or a vacuum regulator 45 (see FIGS. 33-35), and/or providing information (e.g., an alert) on a user interface 49 supported on the cart 34.

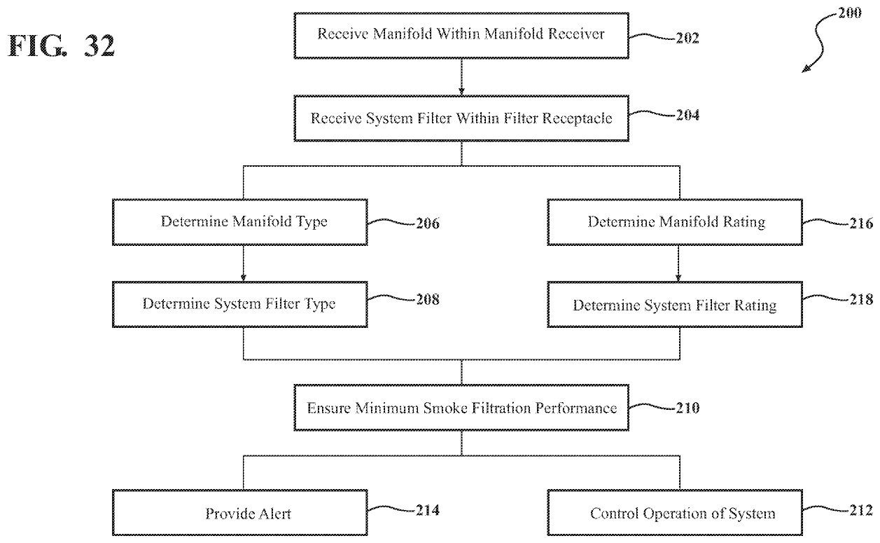

With reference to FIG. 32, an exemplary method includes detecting the manifold 60-70 removably inserted within the manifold receiver(s) 46, 47 (step 202), and detecting the system filter 72-76 removably inserted within the filter receptacle 50 (step 204). One or more processors (e.g., of the controller 54) determines a manifold type based on the manifold identification data (step 206), and determines a system filter type based on the system filter identification data (step 208). The one or more processors, compares a combination of the manifold type and the system filter type against predefined combinations accessed from memory. The predefined combinations are configured assess or ensure a minimum smoke filtration performance (step 210). The minimum smoke filtration performance may be one of a high efficiency particulate air (HEPA) rating and an ultra low particulate air (ULPA) rating, and/or volatile organic compounds (VOC) emissions below a defined threshold.

In certain implementations, the user interface 49 may receive a user input indicative of anticipated usage of the waste collection system 40. Additionally or alternatively, the user input may indicate a desired level of filtration performance such as a filtration rating or a level of VOC emissions. The predefined combinations may be based on the user input. For example, the user may input liquid waste collection only, smoke evacuation only, or a combination thereof. It is understood that should the manifold be detected as not capable of smoke evacuation (e.g., does not include the inlet fitting(s) 108 for the smoke suction tube), the controller may permit operation of the system 40 regardless of the system filter type. The present disclosure also contemplates a computer program product comprising non-transitory computer readable medium configured to be executed by one or more processors to perform the steps of any one of the methods.

For example, the controller of the system 40 may utilize identification data from the identification tags 200 associated with the manifold 60-70 and the system filter 72-76 for responsive, intuitive operation of the system 40. Further, the system filter 72-76, may be an ULPA filter, a HEPA+ filter, or a HEPA filter, and the particulate filter 120 of the manifold 60-70 may be an ULPA filter, a HEPA+ filter, a HEPA filter, and/or include any other suitable filtering media such as one or more absorbers. The HEPA may be EPA-rated HEPA or “true” HEPA. Filtering requirements of surgical smoke evacuation (e.g., regulatory or certification standards) may demand ULPA-level filtration, and it may be desirable to have VOC emissions of the exhausted smoke below certain levels. Therefore, advantages of the present disclosure provide for various combinations of the manifold 60-70 and the system filter 72-76 achieving the necessary filtration performance while minimizing expense to the end user, thereby increasing the likely uptake and resulting safety in the operating theatre. In other words, ULPA-rated filter media is often cost prohibited for single-use components, and therefore the system 40 effectively leverages the presence of two smoke filters (i.e., the manifold 60-70 and the system filter 72-76) to provide ULPA-level filtration and/or minimal VOCs. Non-limiting combinations of the manifold 60-70 and the system filter 72-76 are tabulated in the matrix below:

| | Manifold | | | System Filter | | | |
|---|---|---|---|---|---|---|---|
| Option | HEPA | ULPA | Adsorber | HEPA | ULPA | Adsorber | Result |
| A | No | No | No | Yes | Yes | No | ULPA |
| B | No | No | No | Yes | Yes | Yes | ULPA + VOC |
| C | No | Yes | No | Yes | No | No | ULPA |
| D | No | Yes | Yes | Yes | No | Yes | ULPA + VOC |
| E | Yes | No | No | Yes | No | No | ULPA |
| F | Yes | No | Yes | Yes | No | Yes | ULPA + VOC |
| G | Yes | No | No | Yes | No | Yes | ULPA + VOC |
| H | Yes | No | Yes | Yes | Yes | No | ULPA + VOC |

Therefore, one of the predefined combinations may include the presence of the particulate filter, the absence of the manifold adsorber, the system filter filtration rating being HEPA rated, and the presence of the system filter adsorber. For another example, one of the predefined combinations may include the absence of the particulate filter, the presence of the manifold adsorber, the system filter filtration rating being ULPA rated, and the presence of the system filter adsorber.

In a variant of the method, the one or more processors determine a combined filtration performance based on the manifold filtration rating (step 216) and the system filter filtration rating (step 218). The combined smoke filtration performance may be one of a HEPA rating, an ULPA rating, and/or VOC emissions below a defined threshold.

Should the filtration performance and/or VOC emissions meet a desired criteria, the controller may permit normal operation of the system 40. An indication of optimal filtration performance may be provided on the user interface 49. Conversely, should the filtration performance and/or VOC emissions not meet the desired criteria, the controller may not permit operation of the system 40 (step 212) or an alert may be provided on the user interface 49 (step 214) so the surgeon is aware of the increased risk to suboptimally filtered surgical smoke. For example, the controller 54 may prevent operation of the vacuum source. In such instances, the user interface 49 may be receive an override input from the user in response to the presented alert, after which operation of the vacuum source is permitted.

In certain implementations, the controller 54 may be configured control the vacuum source 41 (and/or the vacuum regulator 45) based on the identification data. The controller may reference a compatibility database to assess the filtration performance and/or VOC emissions of the determined combination of the manifold 60-70 and the system filter 72-76. In particular, the data may identify the manifold as a type only for filtering the liquid medical waste, as a type for filtering the liquid medical waste and the surgical smoke, or as the type only for filtering the surgical smoke. The vacuum level to optimally draw the liquid medical waste through the manifold may be different than the vacuum level to optimally draw the surgical smoke through the manifold. Therefore, based on the data received from the data different types of the manifold, the controller may be configured to operate the vacuum source 41 and/or the vacuum regulator 45 accordingly. For example, the controller may do so to provide a first suction profile based on the manifold type being configured to filter liquid medical waste, a second suction profile based on the manifold type being configured to filter the surgical smoke, a third suction profile based on the manifold type being configured to both the liquid medical waste and the filter surgical smoke. The first suction profile may provide for maximum suction pressure based on a suction level selected by a user, and wherein the second suction profile may be for maximum flow rate. This may be done automatically without requiring input from the user. For example, the system 40 may include a particulate sensor configured to detect the presence of smoke, after which vacuum level may be increased to remove as much smoke as possible while monitoring the thermal constrains on the vacuum source. Of course, the user may provide an input to the user interface 49 to increase, decrease, or otherwise alter the suction profile as desired.

An exemplary arrangement may include the user inserting a first manifold 60, 64, 66, 68, 70 for filtering the liquid medical waste into the manifold receiver 46 associated with the upper waste canister 42, and insert a second manifold 62 for filtering the surgical smoke into the second manifold receiver 47 associated with the lower waste canister 23. The data reader 52 detects the identification tag 198 coupled to the first manifold, and the controller operates the vacuum source 41 and/or the vacuum regulator 45 (e.g., a first vacuum regulator) to independently provide the first suction profile to the upper waste canister 42 in fluid communication with the first manifold. The data reader 53 associated with the lower waste canister 43 detects the identification tag 198 coupled to the second manifold 62, and the controller operates the vacuum source 41 (and/or the vacuum regulator 45) to independently provide the second suction profile to the lower waste canister 43 in fluid communication with the second manifold 62. Other related controls of the vacuum levels based on the manifold type are considered within the scope of the present disclosure.

FIGS. 33-35 schematically illustrate exemplary arrangements of the components of the waste collection system 40 to accommodate the evacuation of the liquid medical waste and the surgical smoke. Solid lines represent the gas flow path(s), and the dashed lines represent the liquid flow path(s). The system 40 includes the vacuum source 41, the vacuum regulator 45, the system filter 72-76, the waste canister 42, and optionally the smoke evacuation unit 48. Conduits (e.g., tubing or piping) operably couple the components. The vacuum regulator 45 is disposed between the waste canister 42 and the vacuum source 41. The system filter 72-76 may be disposed between the vacuum regulator 45 and the vacuum source 41.

Referring to FIG. 33, the manifold 60-70 is configured to be inserted into the manifold receiver 46. The vacuum source 41 is operated to draw the vacuum through the manifold 60 within the manifold receiver 46. The liquid medical waste and the surgical smoke may be simultaneously drawn through the manifold 60 and into the waste canister 42. The liquid medical waste descends under the influence of gravity be collected within the waste canister 42. When desired, the system 40 is operably coupled to a docking station (not shown), after which the collected liquid medical waste is drained through a base of the waste canister 42. The surgical smoke is drawn through an outlet of a cap the waste canister 42, through the vacuum regulator 45, the system filter 72-76, and the vacuum source 41 to be expelled to ambient. In such an arrangement, the surgical smoke has been twice filtered; i.e., within the manifold 60 and the system filter 72-76.

The smoke evacuation unit 48, as shown in FIG. 34, includes a second vacuum source supported on the cart 44, and filtering media (not shown). An exemplary implementation of the smoke evacuation unit 48 is disclosed in the aformentioned International Publication No. WO 2017/112684. The manifold receiver 46 is in fluid communication with the first vacuum source 41 and the second vacuum source. The first vacuum source 41 is operable to draw the liquid medical waste through the manifold 60 to be collected within the waste canister 42. Similar to the previous arrangement, gas entrained within the liquid flow path is drawn through an outlet of the waste canister 42, through the vacuum regulator 45, the system filter 72-76, and the vacuum source 41 to be expelled to ambient. The second vacuum source is operable to draw the surgical smoke through the manifold 60, and the smoke evacuation unit 48 to be expelled to ambient.

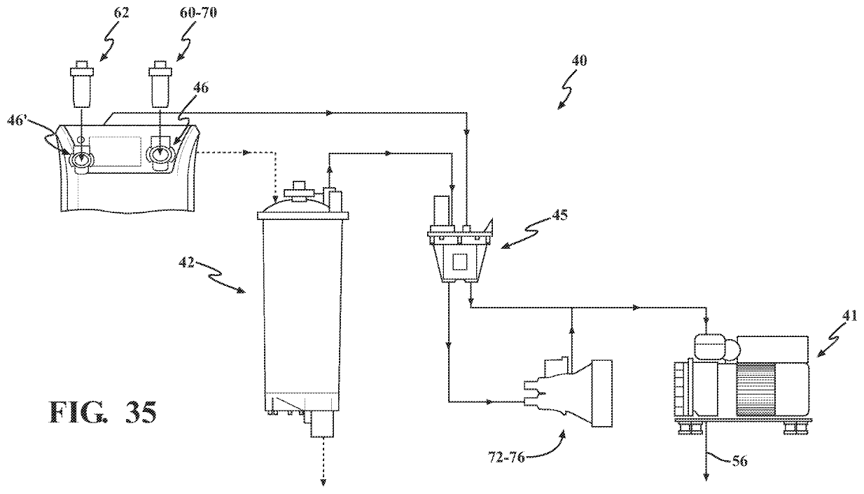

FIG. 35 includes a system 40 in which there is a second manifold receiver 46′. The first manifold receiver 46 may be configured to receive the manifold for filtering liquid medical waste, and the second manifold receiver 46′ may be configured to receive an implementation of the manifold 62 only for filtering the surgical smoke. The second manifold receiver 46′ may be sized or shaped differently than the first manifold receiver 46 such that the user knows which version of the manifold is associated with the respective one of the first and second manifold receivers 46, 46′. Each of the first manifold receiver 46 and the second manifold receiver 46′ may be in fluid communication with the vacuum regulator 45, the system filter 72-76, and the vacuum source 41. The vacuum source 41 is operable to draw the liquid medical waste through the first manifold 60-70 to be collected within the waste canister 42, and to draw the surgical smoke through the second manifold 62. Similar to the previous arrangement, gas entrained within the liquid flow path is drawn through an outlet of the waste canister 42, through the vacuum regulator 45, the system filter 72-76, and the vacuum source 41 to be expelled to ambient. The gas flow path is drawn through the vacuum regulator 45, the system filter 72-76, and the vacuum source 41 to be expelled to ambient. As generally appreciated from FIG. 35, the gas flow path and the liquid flow path are in a parallel arrangement. The arrangements illustrated in FIGS. 33-35 may be implemented on the waste collection system of FIG. 2 including two manifold receivers 46, 47 and two waste canisters 42, 43.

The foregoing disclosure is not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

Certain additional inventive aspects of the disclosure are appreciated from the following exemplary clauses:

Clause 1—A manifold for filtering surgical smoke, the manifold comprising: a trunk defining an outlet opening configured to be operably coupled with a suction system; a head portion coupled to the trunk and comprising an inlet fitting configured to be removably coupled with a smoke suction tube of a smoke evacuation instrument; and a particulate filter supported within the head portion.

Clause 2—The manifold of clause 1, wherein the head portion further comprises a bypass fitting configured to be removably coupled with a fluid suction tube of a suction instrument for fluid drawn therethrough to bypass the particulate filter.

Clause 3—The manifold of clause 1 or 2, wherein the head portion further defines a fluid sump at least partially disposed below a lower wall of the filter casing.

Clause 4—The manifold of any one of clauses 1-4, further comprising a first particulate filter, a second filter media positioned below the first particulate filter, and an impermeable separator disposed between the first particulate filter and the second filter media.

Clause 5—A manifold for a waste collection system, the manifold comprising: a trunk defining an outlet opening configured to be operably coupled with the waste collection system; a head portion coupled to the trunk and comprising an inlet fitting configured to be removably coupled with a smoke suction tube of a smoke evacuation instrument to establish a gas flow path with the outlet opening, a bypass inlet fitting configured to be removably coupled with a fluid suction tube of a suction instrument to establish a liquid flow path with the outlet opening, and a barrier separating the gas flow path and the liquid flow path; and a valve assembly coupled to the head portion and configured to be actuated to prevent and/or establish fluid communication between the outlet opening and at least one of the inlet fitting and the bypass fitting.

Clause 6—The manifold of clause 5, wherein the valve assembly comprises dials each defining at least one opening therethrough, wherein at least one of the dials is rotatable to align or misalign the respective openings.

Clause 7—The manifold of clause 6, wherein the dials are disposed adjacent to one another and between the trunk and the head portion.

Clause 7—The manifold of clause 5, wherein the valve assembly comprises a valving member pivotably disposed within the head portion and configured to be pivoted based on relative strength between the gas flow path and the liquid flow path, and, optionally, wherein the valving member is coupled to the barrier.

Clause 8—The manifold of clause 5, wherein the valve assembly comprises: one or more valving members pivotably disposed within the head portion and biased to a closed state in which fluid communication between the outlet opening and at least one of the inlet fitting and the bypass fitting is prevented; and one or more poppet coupled to the valving members and comprising a distal end positioned proximate to the inlet fitting and the bypass fitting, wherein the poppets are configured to actuate a respective one of the valving members to an open position in response to a corresponding one of the inlet fitting and the bypass fitting receiving a coupler.

Clause 9—A waste collection system comprising: a cart; a first vacuum source supported on the cart; a second vacuum source supported on the cart; a waste canister in fluid communication with the first vacuum source; a vacuum regulator in fluid communication and positioned between the first vacuum source and the waste canister; an exhaust; and a manifold receiver in fluid communication with the first vacuum source and the second vacuum source, wherein the manifold receiver is configured to removably receive a manifold, wherein the first vacuum source is operable to draw liquid medical waste through the manifold to be collected within the waste canister, and wherein the second vacuum source is operable to draw surgical smoke through the manifold to be exhausted from the exhaust in a manner that bypasses the waste canister.

Clause 10—A waste collection system comprising: a cart; a vacuum source supported on the cart; a waste canister in fluid communication with the vacuum source; a first manifold receiver in fluid communication with the vacuum source, wherein the first manifold receiver is configured to removably receive a first manifold for filtering liquid medical waste; a second manifold receiver in fluid communication with the vacuum source, wherein the second manifold receiver is configured to removably receive a second manifold for filtering surgical smoke; and wherein the vacuum source is operable to draw the liquid medical waste through the first manifold and the first manifold receiver to be collected within the waste canister, and draw the surgical smoke through the second manifold and the second manifold receiver.

Clause 11—The waste collection system of clause 10, further comprising a vacuum regulator in fluid communication with the vacuum source and the first and second receivers.

Clause 12—A waste collection system comprising: a cart; a vacuum source supported on the cart; a waste canister in fluid communication with the vacuum source; and a receiver in fluid communication with the vacuum source, wherein the receiver is configured to removably receive a manifold; a vacuum regulator in fluid communication with the vacuum source and the receiver; and a data reader configured to detect an identification tag coupled to the manifold to determine a manifold type from identification data from the identification tag, a controller in electronic communication with the vacuum source, the vacuum regulator, and the data reader, wherein the controller is configured to operate the vacuum source and/or the vacuum regulator based on the manifold type as determined.

Clause 13—The waste collection system of clause 12, wherein the controller is configured to operate the vacuum source and/or the vacuum regulator to provide a first suction profile based on the manifold type being configured to filter liquid medical waste, operate the vacuum source and/or the vacuum regulator to provide a second suction profile based on the manifold type being configured to filter surgical smoke, and operate the vacuum source and/or the vacuum regulator to provide a third suction profile based on the manifold type being configured to both the liquid medical waste and the filter surgical smoke.

Clause 14—The waste collection system of clause 13, wherein the first suction profile is configured to provide for maximum suction pressure based on a suction level selected by a user, and wherein the second suction profile is configured to provide for maximum flow rate.

1. A manifold for filtering surgical smoke, the manifold comprising:
a trunk defining an outlet opening configured to be operably coupled with a suction system;
a head portion coupled to the trunk and comprising an inlet fitting configured to be removably coupled with a smoke suction tube of a smoke evacuation instrument; and
a filter assembly disposed within the head portion and comprising a filter casing defining a casing inlet and a casing outlet, wherein the filter assembly further comprises a particulate filter supported within the filter casing between the casing inlet and the casing outlet.

2. The manifold of claim 1, wherein the filter casing is vertically offset relative to the trunk, wherein the head portion further comprises a bypass fitting positioned above or below the filter casing and opening into the trunk, and wherein the bypass fitting is configured to be removably coupled with a fluid suction tube of a suction instrument for fluid drawn therethrough to bypass the particulate filter.

3. The manifold of claim 1, wherein the head portion further defines a fluid sump at least partially disposed below a lower wall of the filter casing.

4. The manifold of claim 3, wherein the head portion comprises a downwardly extending protrusion defining a portion of the fluid sump.

5. The manifold of claim 3, wherein the filter assembly further comprises a seal positioned between the lower wall of the filter casing and an interior surface of the head portion, wherein the fluid sump extends beneath the filter casing between a cap faceplate and the seal.

6. The manifold of claim 1, wherein the filter casing comprises a distal barrier defining the casing inlet, and a port extending distally from the distal barrier and positioned within the head portion.

7. The manifold of claim 6, wherein an axis defined by the inlet fitting is positioned below an axis defined by the port.

8. The manifold of claim 6, wherein a proximal end of the inlet fitting is positioned proximal to a distal end of a port of the filter casing.

9. The manifold of claim 1, wherein the head portion comprises a proximal barrier defining an aperture providing fluid communication between the filter assembly and the trunk, wherein the aperture is positioned between the casing outlet and the outlet opening.

10-13. (canceled)

14. The manifold of claim 1, further comprising a filter basket disposed within the trunk.

15-25. (canceled)

26. A manifold for filtering surgical smoke, the manifold comprising:
a trunk defining an outlet opening configured to be operably coupled with a suction system;
a head portion coupled to the trunk and defining a bore; and
a filter assembly disposed within the head portion and comprising a filter casing, and a particulate filter disposed within the filter casing, wherein the filter casing comprises a distal barrier, an inlet fitting extending distally from the distal barrier and through the bore of the head portion, wherein the inlet fitting configured to be removably coupled with a coupler of a smoke suction tube.

27. The manifold of claim 26, wherein the head portion further comprises a rim defining a cavity sized to receive the coupler of the smoke suction tube, wherein the bore is defined at a base of the cavity.

28. The manifold of claim 27, wherein the rim and the inlet fitting are concentrically arranged.

29. The manifold of claim 26, wherein the bore is a first bore and the inlet fitting is a first inlet fitting, and wherein the head portion defines a second bore and the filter casing comprises a second inlet fitting extending distally from the distal barrier and through the second bore of the head portion.

30. The manifold of claim 29, wherein the first and second bores are in a side-by-side arrangement.

31. A manifold for filtering surgical smoke, the manifold comprising:
a trunk defining an outlet opening configured to be operably coupled with a suction system;
a head portion coupled to the trunk and comprising a rim defining a cavity sized to receive a coupler of a smoke suction tube, wherein a bore is defined at a base of the cavity; and
a filter assembly disposed within the head portion and comprising a filter casing, and a particulate filter disposed within the filter casing, wherein the filter casing comprises a distal barrier and defines an inlet port within the distal barrier and aligned with the bore of the head portion, wherein the inlet port is configured to be removably coupled with the coupler of the smoke suction tube with the coupler disposed within the cavity.

32. The manifold of claim 31, wherein the rim and the inlet fitting are concentrically arranged.

33. The manifold of claim 31, wherein the rim is a first rim, the cavity is a first cavity, the bore is a first bore, and the inlet port is a first inlet port, and wherein the head portion comprises a second rim defining a second cavity sized to receive another coupler of another smoke suction tube, wherein a second bore is defined at a base of the second cavity.

34. The manifold of claim 33, wherein the first and second rims are in a side-by-side arrangement.

35. The manifold of claim 26, wherein the head portion further includes at least one bypass fitting opening into the trunk and configured to be removably coupled with a fluid suction tube of a suction instrument for fluid drawn therethrough to bypass the particulate filter.

36-71. (canceled)
